(19) United States
(12) Patent Application Publication
(10) Pub. No.: US 2020/0330749 A1
(43) Pub. Date: Oct. 22, 2020

(54) INTRACALVARIAL BCI SYSTEMS AND METHODS FOR THEIR MAKING, IMPLANTATION AND USE

(71) Applicant: Inner Cosmos LLC, New Haven, CT (US)

(72) Inventors: Meron GRIBETZ, New York City, NY (US); Eric Clade LEUTHARDT, St. Louis, MO (US); Daniel W. MORAN, Ballwin, MO (US)

(73) Assignee: Inner Cosmos LLC, New Haven, CT (US)

(21) Appl. No.: 16/954,554

(22) PCT Filed: Dec. 27, 2018
(86) PCT No.: PCT/IB2018/060667

§ 371 Date: Jun. 17, 2020

Related U.S. Application Data
(60) Provisional application No. 62/611,043, filed on Dec. 28, 2017.

(51) Int. Cl. A61N 1/05; A61N 1/36; A61N 1/375

(52) U.S. Cl. CPC A61N 1/0531; A61N 1/36096; A61N 1/37514; A61N 1/3752; A61N 1/36085; A61N 1/37518; A61N 1/36064

(57) ABSTRACT

An intra-calvarial implant (ICI) includes one or more electrodes for sensing electrical signals from a brain of a mammal and for electrically stimulating one or more regions of the brain. The electrode(s) are implanted between an outer table and an inner table of the calvarial bone without fully penetrating the inner table, at least one reference electrode, an electronic circuitry module operatively connected to the one or more electrode(s) and to the reference electrode and for controlling the sensing and the stimulating, for at least partially processing the sensed electrical signals to obtain data for storing the data and for wirelessly transmitting the data to an external receiver. The ICI also includes a power harvesting device suitably electrically connected to one or more components of the electronic circuitry module of the ICI for energizing one or more components of the ICI. An ICI system includes ICI(s) and an external controller.

This application claims the benefit of priority under 35 USC § 119(e) of U.S. Provisional Patent Application No. 62/611,043 filed on 28 Dec. 2017, the contents of which are incorporated herein by reference as if fully set forth herein in its entirety.

### FIELD AND BACKGROUND OF THE INVENTION

The present invention, in some embodiments thereof, relates to the field of brain computer interface systems (BCIs), and more particularly to Intracalvarial BCIs including electrodes and electrode arrays for implantation within the calvarial bone of a skull for recording electrical activity from brain tissues underlying the calvarial bone and/or for electrically stimulating brain tissues underlying the calvarial bone.

Recording of electrical signals from the cortical surface of the brain and electrically stimulating selected cortical regions as well as other deeper brain regions underlying the cortex may enable neuro-modulation of brain electrophysiology that may have a wide range of clinical and non-clinical applications. In some clinical applications, cortical stimulation may be used to modify cortical excitability to treat numerous neuropsychiatric diseases such as, among others, depression, ADHD, OCD, addiction, and obesity.

The recording of cortical electrical signals may also be implemented in brain computer interfaces that may be used to treat a wide array of motor disabilities.

Brain recording and/or stimulating methods may also be used for modulating the brain physiology to enhance cognitive function in healthy individuals or to improve cognitive function in some patients having neuropsychiatric diseases affecting cognitive performance such as, inter alia, depression, ADHD, OCD, Various eating disorders, epilepsy, and many other psychiatric, neurodegenerative, neurological and neuropsychiatric disorders.

Depending on the stimulation modality, the brain region being stimulated, and the interface regime, cognitive operations such as attention, memory, analytic abilities, and mood may all be enhanced beyond a given individuals normal baseline.

Several types of brain recording/stimulation are currently known. The least invasive recording method is EEG. In this method recording electrodes are applied externally to the skull of the patient without penetrating the skin of the scalp.

While EEG has the advantage of being a non-invasive low risk recording method, it has several problems resulting from the substantial distance of the recording electrodes from the source of the electrical signals in the cortex and from the intervening skin and bone tissues. Such problems may include a relatively low signal to noise ratio (SNR) due to the low signal amplitude (typically in the range of 10-100 μV) and substantial attenuation of the higher frequency range part of the cortical signal by the intervening bone and skin tissues disposed between the recording EEG electrodes and the cortical surface, resulting. As a result of this frequency attenuation, the part of the cortical electrical activity in the gamma frequency band (f≥30 Hz) may be severely attenuated or completely lost below the noise floor in such EEG recordings, often resulting in loss of physiologically relevant information.

Other disadvantages of EEG recording methods and devices include the need for good electrical contact between the surface of the scalp and the EEG electrodes. The electrically conducting gels or preparations used to achieve and maintain such electrical contact are often messy to apply, may cause patient discomfort, are difficult to hold and maintain in the same position, and may change their resistance or impedance due to drying or loss of water content during extended periods of use that may result in degrading or altering their performance. Additionally, EEG electrode assemblies may not be suitable for long term ambulatory or permanent use because they are cumbersome, are very visible on the patient's head (which may result in the patient's reluctance to use them for long term extended periods of time).

Calvarial screws have been used for either sensing/recording electrical brain signals or for performing transcranial electrical stimulation (TES). For example, Watanabe et al. describe using two calvarial screws for performing TES in an article entitled “Transcranial electrical stimulation through screw electrodes for intraoperative monitoring of motor evoked potentials” published in J. Neurosurg 100: pp 155-160 (2004). Similarly, screw electrodes have been used for sensing electrical brain activity in animals. However, in all these cases, the upper part of the screw protrudes through the upper table of the skull bone in order to allow connecting a lead to the upper part for performing the sensing or the stimulation. The part of the screw protruding out of the skull bone may operate as an antenna and may disadvantageously pick up extraneous electrical noise which may decrease the SNR. Thus, the use of such calvarial screws, while adequate for experimental use in animals or for short term intraoperative use in neurosurgery patients, may not be suitable for long term use in ambulatory patients due to the possibility of noise pickup in changing environmental conditions which the patient may be exposed to. Moreover, long term use of calvarial screws may be objected to by patients due to aesthetic reasons and the need to be connected to stimulating and/or sensing leads which may be cumbersome.

Recently, a new method for non-invasive trans-cranial stimulation of brain tissues was disclosed in a paper by Nir Grossman, David Bono, Nina Deric, Suhasa B. Kodandaramalah, Andrii Rudenko, Ho-Jun Suk, Antonino M. Cassara, Esra Neufeld, Niels, Li Huei Tsai, Alvaro Pascual-Leone and Edwards S. Boyden, “Non-Invasive Deep Brain Stimulation via Temporally Interfering Electric Fields”, Cell 169, pp. 1029-1041, Jun. 1, 2017.

This non-invasive method enables stimulation of selected brain regions using extra-cranial electrodes similar to sensing EEG electrodes to apply oscillating electric fields at two slightly different frequencies to the brain, resulting in an electric field envelope that oscillates at the beat frequency of the two different frequencies. The two different frequencies are at a relatively high frequency range (typically above 1 KHz) so, each frequency by itself cannot cause neuronal stimulation by themselves. This method allows the non-invasive stimulation of deep brain structures by generating a defined region of electrical fields that oscillates at the beat frequency enabling neuronal recruitment within a selected location by electrical stimulation of neurons and/or neuronal parts within the recruitment region. The method of Grossman et al. is referred to hereinafter as trans-cranial frequency interference stimulation (TFIS).

However, TFIS may share some of the problems of using EEG sensing methods because it uses stimulating electrodes similar to EEG electrodes that are applied to the surface of the skull for stimulation. Such common problems may include, electrode instability, electrical coupling to the scalp necessitating electrically conductive gels or formulations, the sensitivity of scalp electrodes to accidental dislodgement or movement and/or to changes in electrical electrode impedance due to changes in the hydration state of such electrically conductive gels or formulations. Such problems may make the use of EEG type scalp electrodes undesirable for long term use such as in ambulatory patients.

Another, more invasive method for performing cortical surface recording uses epidural or subdural surface electrodes (typically, electrode arrays) which are placed on the surface of the dura mater (epidural Ecog) or on the cortical surface (subdural Ecog) to record an Electrocorticogram (Ecog) signal. Typically, the amplitude of the sense/recorded Ecog signals is about 10-20 mV. Such methods have the advantage of resulting in better signal to noise ratio (SNR) and enabling a higher frequency range of sensing due to their close proximity to the cortical surface. While intracranial Ecog arrays solve many of the problems of EEG recording techniques, a fundamental barrier for more wide spread adoption of such intra-cranially implanted Ecog electrode arrays is the invasiveness of the implantation of the electrodes. Once the skull and dura mater are penetrated with either intra-parenchymal or electrocorticographic electrodes there is a risk of having an intracranial hemorrhage or infection that could cause major harm, morbidity, or even death. While these risks, generally speaking, are very small the fact they exist substantially changes a patient's perception of considering adoption. This also changes the manner in which patients are treated by physicians after implantation. If an intracranial electrode implant is surgically placed (e.g. deep brain stimulator, cortical stimulator, etc), at the very least the patients are kept overnight for observation in a hospital to ensure that, should an intracranial complication arise, it can be rapidly addressed. This increases the cost of such intracranial implantation procedures and may limit their use for many applications

There is therefore a long felt need for a recording and/or stimulating brain interface that has no or little risk of intracranial complications, may be more esthetic and have a lesser implantation impact yet still be able to record and/or stimulate the brain with a functional equivalence that is close to that of intracranial Ecog devices.

### SUMMARY OF THE INVENTION

There is therefore provided, in accordance with some embodiments of the present application, an intra-calvarial implant (ICI). The ICI includes one or more electrodes for sensing electrical signals from a brain of a mammal and for electrically stimulating one or more regions of the brain. The one or more electrodes are configured to be implanted between an outer table and an inner table of the calvarial bone without fully penetrating the inner table. The ICI also includes at least one reference electrode and an electronic circuitry module operatively connected to the one or more electrodes and to the at least one reference electrode and configured for controlling the sensing and the stimulating, for at least partially processing sensed electrical signals to obtain data for storing the data and for wirelessly transmitting the data to an external receiver. The ICI also includes a power harvesting device suitably electrically connected to one or more components of the electronic circuitry module of the ICI for energizing components of the ICI.

In accordance with some embodiments, the one or more electrodes are attached to an electrode supporting member.

In accordance with some embodiments, the electrode supporting member is selected from, a housing wherein the one or more electrodes are attached to the housing or a part thereof, the housing sealingly includes the electronic circuitry module therein, an electrode supporting member separate from a housing of the ICI wherein the one or more electrodes are attached to the electrode supporting member and electrically connected to the electronic circuitry module that is sealingly disposed within the housing, an elongated lead-like flexible electrode supporting member attached to a housing sealingly enclosing the electronic circuitry module, wherein the one or more electrodes are attached to the elongated flexible electrode member, and

a flexible electrode array supporting member electrically connected to a housing sealingly enclosing the electronic circuitry module.

In accordance with some embodiments, the electronic circuitry module is selected from, an electronic circuitry module sealing enclosed in a housing, the housing is disposed in it's entirety between the outer table and the inner table of the calvarial bone, an electronic circuitry module sealingly enclosed within a housing, wherein a part of the housing is disposed between the outer table and the inner table and another part of the housing is disposed outside the outer table, and

an electronic circuitry module sealingly enclosed within a housing disposed outside an outer surface of the outer table.

In accordance with some embodiments, the one or more electrodes are disposed in one or more hollow passages formed within a cancellous bone layer of the calvarial bone.

In accordance with some embodiments, one or more of the one or more electrodes and the electronic circuitry module are disposed within a chamber formed within a cancellous bone of the calvarial bone.

In accordance with some embodiments, the chamber includes a recess formed within the inner table of the calvarial bone without fully penetrating the inner table.

In accordance with some embodiments, The ICI according to any of claims 6-7, wherein the chamber has a chamber opening formed within the outer table, the chamber opening has a cross sectional area and wherein the chamber is selected from, a chamber in which at least one cross-sectional area of the chamber taken parallel to the cross sectional area of the opening is larger than the cross-sectional area of the chamber opening, a chamber in which at least one cross-sectional area of the chamber taken parallel to the cross sectional area of the opening is equal to the cross-sectional area of the opening, a chamber in which at least one cross-sectional area of the chamber taken parallel to the cross sectional area of the opening is smaller than the cross-sectional area of the opening, and

a chamber having at least one laterally extending elongated hollow passage formed within the cancellous bone.

In accordance with some embodiments, the one or more electrodes are selected from the list consisting of, multiple single electrodes, a single electrode array, multiple electrode arrays, a flexible electrode array, a foldable electrode array, a rigid electrode array, a planar electrode array, a linear electrode array, a flexible linear electrode array, a curved surface electrode array electrode array, an electrocorticography (Ecog) electrode array and any non-mutually exclusive combinations thereof.

In accordance with some embodiments, the electronic circuitry module is selected from,

an electronic circuitry module programmed for wirelessly receiving control signals from an external control unit, selecting a subset of electrodes from the one or more electrodes responsive to the received control signals, sensing electrical brain signals through the selected electrode(s), conditioning and/or amplifying the signals and wirelessly communicating the signals to an external receiver, an electronic circuitry module programmed for wirelessly receiving control signals from an external control unit, selecting a subset of electrodes from the one or more electrodes responsive to the received control signals and stimulating through the selected subset of electrodes one or more regions of the brain, an electronic circuitry module programmed for wirelessly receiving control signals from an external control unit, selecting a first subset of electrodes from the one or more electrodes responsive to the received control signals, sensing electrical brain signals through the first subset of electrodes, conditioning and/or amplifying the signals and wirelessly communicating the signals to an external receiver, wirelessly receiving control signals from an external control unit, selecting a second subset of electrodes from the one or more electrodes responsive the control signals and stimulating through the second subset of electrodes one or more regions of the brain, and

an electronic circuitry module programmed for selecting a first subset of electrodes from the one or more electrodes, sensing electrical brain signals through the selected first subset of electrodes, conditioning and/or amplifying the signals, processing the signals to detect an indication associated with a neurological and/or neuropsychiatric and/or psychiatric state of the brain, selecting a second subset of electrodes from the one or more electrodes based on the indication and stimulating through the second subset of electrodes one or more regions of the brain.

In accordance with some embodiments, the first subset and the second subset are selected from,

the first subset is different than the second subset, and

the first subset is identical to the second subset.

In accordance with some embodiments, the power harvesting device is selected from an ultrasonic energy harvesting device and an electromagnetic power harvesting device.

In accordance with some embodiments, the power harvesting device is selected from a power harvesting device comprising an induction coil and a power harvesting device comprising a piezoelectric transducer.

In accordance with some embodiments, at least one part of the power harvesting device is disposed under or within the scalp of the mammal.

In accordance with some embodiments, at least one part of the power harvesting device is disposed within the ICI.

In accordance with some embodiments, the electrode supporting member has a shape selected from, a disc-like shape, a cylindrical shape, a shape having an ellipsoidal cross section, a shape having a polygonal cross section, and a shape having an irregular cross section.

In accordance with some embodiments, the electrode supporting member is configured as an openable and closable housing having an openable and closable compartment therein.

In accordance with some embodiments, the electronic circuitry module is disposed within the compartment.

In accordance with some embodiments, the mammal is selected from, a non-human mammal, and a human patient.

In accordance with some embodiments, the at least one reference electrode is selected from, at least one reference electrode disposed between the outer table and the inner table of the calvarial bone without fully penetrating the inner table, and

at least one reference electrode disposed on the electrode supporting member,

at least one reference electrode dispose within the outer table or on the outer surface of the outer table,

at least one reference electrode disposed between the outer surface of the outer table and the scalp, and

at least one reference electrode disposed under or within the scalp.

In accordance with some embodiments, the at least one reference electrode is selected from,

at least one reference electrode attached to the electrode supporting member,

at least one reference electrode attached to the housing or to any parts thereof,

at least one reference electrode attached to an electrode supporting member separate from a housing of the ICI,

at least one reference electrode attached to an elongated lead-like flexible electrode supporting member attached to a housing sealingly enclosing the electronic circuitry module, wherein the one or more electrodes are attached to the elongated flexible electrode member, and

at least one reference electrode attached to a flexible electrode array electrically connected to a housing sealingly enclosing the electronic circuitry module.

There is also provided, in accordance with some embodiments of the systems of the present application, an ICI system The system includes: One or more of the intra-calvarial implant (ICI) for sensing electrical activity in a brain and/or for stimulating the brain, and an external controller unit configured for wirelessly bidirectionally communicating with the one or more ICI to receive and/or transmit signals to the one or more ICI.

In accordance with some embodiments of the system, the external controller is configured for wirelessly receiving data and/or signals wirelessly transmitted by the one or more ICI, processing the received data and/or signals and for wirelessly transmitting data and/or control signals to the one or more ICI responsive to the processing.

In accordance with some embodiments of the system, the one or more ICI include two or more ICIs and the control signals include signals for synchronizing the operation of the two or more ICI.

In accordance with some embodiments of the system, the one or more ICI include two or more ICIs. At least one ICI of the two or more ICIs is a master ICI programmed to wirelessly bidirectionally communicate with the external controller and to wirelessly communicate with the remaining ICIs of the two or more ICIs to receive signals from the remaining ICIs and to transmit control signals to the remaining ICIs for controlling the operation of the remaining ICIs. The remaining ICIs are slave ICIs programmed to communicate only with the master ICI.

In accordance with some embodiments of the system, the master ICI is programmed to control the timing of stimulation of one or more brain regions by the one or more ICIs.

In accordance with some embodiments of the system, the external controller unit is programmed to wirelessly receive signals and/or data from all the ICIs of the one or more ICIs and to wirelessly control the operation of all the ICIs of the one or more ICI.

In accordance with some embodiments of the system, the system also includes a power transmitting device for transmitting power to power harvesting device(s) of the one or more ICI.

In accordance with some embodiments of the system, the power transmitting device is integrated into the external controller.

In accordance with some embodiments of the system, the mammal is a human patient and wherein the external controller is an external controller having an application operating thereon for controlling the operation of the system.

In accordance with some embodiments of the system, the application is programmed to perform one or more steps selected from, bidirectionally exchanging data and/or control signals with at least some ICIs of the one or more ICI, processing data received from the at least some ICIs, and interacting with the human in which the one or more ICI are implanted using a user interface included in the external controller to receive data from the human user indicative of a clinical condition and/or neurophysiological condition and/or neuropsychiatric condition of the patient, processing the data received from the patient and controlling the stimulating of the brain of the patient based on the received and/or processed data.

In accordance with some embodiments of the system, the application is programmed to perform one or more steps selected from, bidirectionally exchanging data and/or control signals with a ICI of the one or more ICI, at least some of the data comprises data communicated to the master ICI from the remaining ICIs of the one or more ICI, processing data received from the master ICI, using a user interface included in the external controller to receive data from the human user indicative of a clinical condition and/or neurophysiological condition and/or neuropsychiatric condition of the patient, processing the data received from the patient and controlling the stimulating of the brain of the patient based on the received and/or processed data.

There is also provided, in accordance with some embodiments of the methods of the present application, a method for implanting an intra-calvarial implant (ICI) in a mammal, the method includes the steps of:

making an incision in a scalp of the mammal,

exposing a region of the calvarial bone of the mammal,

forming one or more openings in an outer table of the calvarial bone,

forming one or more hollow passages in the cancellous bone of the calvarial bone without

fully penetrating an inner table of the calvarial bone,

inserting at least part of the ICI into the one or more hollow passages such that the one or more electrodes of the ICI are disposed between the outer table and the inner table of the calvarial bone, and

arranging the scalp to cover the exposed region of the scalp.

In accordance with some embodiments of the method, the first step of forming is performed by drilling and/or burring.

In accordance with some embodiments of the method, the second step of forming is performed by drilling and/or burring and/or laser ablating.

In accordance with some embodiments of the method, the one or more opening is a single opening, and wherein the method also includes after the step of inserting, the step inserting a shim into the opening in the outer table such that an upper surface of the shim is flush with an outer surface of the outer table.

In accordance with some embodiments of the method, the step of inserting also includes sealingly attaching the ICI to the calvarial bone.

In accordance with some embodiments of the method, the step of inserting also includes sealingly attaching the shim to the calvarial bone.

In accordance with some embodiments of the method, the second step of forming comprises forming a hollow chamber within a cancellous bone and forming or more additional hollow passages extending laterally from the hollow chamber for receiving therein one or more elongated flexible electrode carrying members.

In accordance with some embodiments of the method, the method also includes the step of electrically connecting an induction coil to an electronic circuit module included in the ICI and disposing the induction coil between the outer table and the scalp.

In accordance with some embodiments of the method, the method also includes the step of thinning one or more regions of the inner table underlying the one or more passages to form one or more regions of the inner table having a reduced bone thickness therein to increase the amplitude of brain electrical signals sensed by the one or more electrodes.

In accordance with some embodiments of the method, the sealingly attaching comprises sealingly attaching by a biocompatible sealing material.

In accordance with some embodiments of the method, the second step of forming includes forming a first hollow passage beginning at the opening and extending towards the inner table in a direction substantially orthogonal to the outer table, and forming at least one second hollow passage within the cancellous bone, the at least second hollow passage is contiguous with the first hollow passage and extends laterally from the first hollow passage.

In accordance with some embodiments of the method, the one or more electrodes of the ICI are selected from the list consisting of, a single electrode, multiple single electrodes, a single electrode array, multiple electrode arrays, a flexible electrode array, a foldable electrode array, a rigid electrode array, a planar electrode array, a linear electrode array, a flexible linear electrode array, a curved surface electrode array electrode array, an electrocorticography (Ecog) electrode array and any non-mutually exclusive combinations thereof.

### BRIEF DESCRIPTION OF THE SEVERAL VIEWS OF THE DRAWINGS

Some embodiments of the invention are herein described, by way of example only, with reference to the accompanying drawings, in which like components are designated by like reference numerals. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic, part cross-sectional diagram illustrating a part of a calvarial bone and the scalp;

FIG. 2 is a schematic side view illustrating a human skull and some of the calvarial bones thereof;

FIGS. 3-4 are schematic part cross-sectional diagrams, illustrating two different ICIs, in accordance with some embodiments of the ICIs of the present application;

FIG. 5 is a schematic part cross-sectional view, illustrating an electrode assembly having obliquely arranged elongated members carrying electrodes, in accordance with some embodiments of the ICIs of the present application;

FIG. 6 is a schematic cross sectional view illustrating an electrode assembly having bent elongated members including multiple electrodes, in accordance with some embodiments of the ICIs of the present application;

FIG. 7 is a schematic bottom view of the electrode assembly of FIG. 6;

FIG. 8 is a schematic part cross-sectional view illustrating an electrode assembly having a single extended linear electrode carrying member having multiple electrodes therein, in accordance with some embodiments of the ICIs of the present application;

FIG. 9 is a schematic isometric view illustrating a skull with an implanted intra-calvarial electrode assembly having an extended flexible electrode carrying member with multiple electrodes extending intra-cranially within the calvaria, in accordance with some embodiments of the ICIs of the present application;

FIG. 10 is a schematic, part cross-sectional view of a “monoblock type” electrode assembly having an integrated electrode array and implanted within a calcarial bone, in accordance with some embodiments of the ICIs of the present application;

FIG. 11 is a schematic bottom view of the electrode assembly of FIG. 10;

FIG. 12 is a schematic, part cross-sectional view of another “monoblock type” electrode assembly having an integrated electrode array disposed within a recess made in the inner table of a calvarial bone, in accordance with some embodiments of the ICIs of the present application;

FIG. 13 is a schematic part cross-sectional view of a system including an intra-calvarial electrode array, an electronic circuitry module and a power harvesting coil disposed on the outer table of a calvarial bone, in accordance with an embodiment of the systems of the present application.

FIGS. 14A-14E are schematic part cross-sectional views illustrating various steps of a method for forming extended laterally oriented hollow passages and/or hollow chambers within the cancellous bone layer of a calvarial bone, in accordance with some methods of implantation of the ICIs and systems of the present application;

FIG. 15 is a schematic block diagram illustrating some of the components of an intra-calvarial system for sensing and/or recording brain electrical signals, in accordance with some embodiments of the systems of the present application;

FIG. 16 is a schematic block diagram illustrating some of the components of an intra-calvarial system for stimulating one or more regions of the brain, in accordance with some embodiments of the systems of the present application;

FIG. 17 is a schematic block diagram illustrating some of the components of an intra-calvarial system for sensing/recording brain electrical signals and for stimulating one or more regions of the brain, in accordance with some embodiments of the systems of the present application;

FIG. 18 is a schematic block diagram of an exemplary system for sensing/recording brain electrical signals and for stimulating one or more regions of the brain, in accordance with some embodiments of the systems of the present application;

FIG. 19 is a schematic functional block diagram, illustrating some components devices of a hand held device or a portable device usable as part of the systems of the present application;

FIG. 20 is a schematic block diagram illustrating some components of an energizing device usable for providing power to some of the systems and/or ICIs of the present applications;

FIG. 21 is a schematic cross-sectional diagram illustrating an intra-calvarial implant that is fully disposed within the cancellous bone layer of the calvarial bone of the skull, in accordance with some embodiments of the implants of the present application;

FIG. 22 is a schematic isometric view illustrating a system including four ICIs implanted in the skull of a patient in accordance with some embodiments of the systems of the present application;

FIG. 23 is a schematic functional block diagram illustrating the communication scheme of a first configuration of the system components of FIG. 22, in accordance with some embodiments of the systems of the present application; and

FIG. 24 is a schematic functional block diagram illustrating the communication scheme of a second configuration of the system components of FIG. 22, in accordance with some embodiments of the systems of the present application.

### DETAILED DESCRIPTION OF SPECIFIC EMBODIMENTS OF THE INVENTION

### Abbreviations

The following abbreviations are used throughout the specification and the claims of the present application:

| Abbreviation | Means |
|---|---|
| μV | microvolt |
| CT | Computerized tomography |
| CVD | Chemical vapor deposition |
| DCES | Direct cortical electrical stimulation |
| Ecog | Electrocorticography |
| EEG | Electroencephalography |
| FI | Frequency interference |
| fMRI | Functional magnetic resonance imaging |
| ICE | Intra-calvarial Electrode |
| ICEA | Intra-calvarial electrode assembly |
| ICI | Intra-calvarial Implant |
| MRI | Magnetic resonance imaging |
| mV | millivolt |
| SNR | Signal to Noise Ratio |
| TES | Trans-cranial electrical stimulation |
| TFIS | Trans-cranial frequency interference stimulation |
| TMS | Transcranial Magnetic Stimulation |

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Before explaining at least one embodiment of the invention in detail, it is to be understood that the invention is not necessarily limited in its application to the details of construction and the arrangement of the components and/or methods set forth in the following description and/or illustrated in the drawings and/or the examples. The invention is capable of other embodiments or of being practiced or carried out in various ways. It is expected that during the life of a patent maturing from this application many relevant types of electrodes and electrode arrays will be developed and the scope of the terms “electrode” and “electrode array” are intended to include all such new technologies a priori. As used herein the term “about” refers to ±10%. The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application and claims, the term “plurality” means “two or more”.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The present application discloses intra-calvarial electrodes, intra-calvarial electrode arrays and ICIs that are implanted within the calvarial bone of the skull. Also disclosed are methods of implanting such electrodes within the calvarial bone and methods for using the intra-calvarial electrode(s) for sensing cortical electrical activity using the intra-calvarial electrodes and methods of stimulating various different brain regions using intra-calvarial electrodes and electrode arrays,

It is noted that the terms “calvarial bone”, “calvarial bones” and “Calvaria” and their conjugate and plural forms are interchangeably used throughout the specification and the claims of the present application to include one or more of the following bones or parts of the following bones: the frontal bone, the left and right parietal bones, the occipital bone, the non-squamous parts of the left and right temporal bones, and the occipital bone. The terms “calvarial bone” and its plural and conjugate forms may also mean any combination of two or more calvarial bones or parts of bones selected from the above list and fused together. It is however noted that the term “calvarial bone” and its conjugate and plural forms, as used in the specification and the claims also includes any bone of the skull in which the ICIs of the present application may be implanted.

To achieve the least amount of invasiveness, while still enabling substantial stimulation and recording capabilities, the ICIs disclosed in the present application have electrodes that are implanted into the calvarial bone but do not fully penetrate the inner table of the calvarial bone. Important barriers that prevents infection and blood from affecting the brain are the dura mater and the inner table of the skull bones. If these barriers are not compromised, penetrated, or affected, then should there be some bleeding or infection related to the implant there would be no impact on the brain itself. This unique position of the electrodes also confers significant recording advantages. By implanting such electrodes or electrode arrays within the skull between the outer table and the inner table of the calvarial bone and close to the inner table of the calvarial bone (but without fully penetrating the inner table and passing through it) focal cortical signals may be readily detected since the electrode is approximately 2-3 millimeters from the cortical surface.

The electrical signals sensed and recorded in this way would be somewhat similar to electrocorticography (Ecog) in which electrodes are placed on the surface of the brain. Ecog signals have been shown to have substantial advantages in SNR in that they can detect focal cortical changes and record higher frequencies than typical EEG recording techniques (such as, for example, gamma rhythms). Numerous publications have shown the information value of Ecog signals for brain computer interfaces and for identifying highly resolved cortical dynamics related to cognition (motor, language, attention, memory, vision, etc). The disclosed intra-calvarial electrode(s), ICIs and the systems including such electrodes and/or ICIs, are able to sense/record a similar level of signal quality without the risk of intracranial implantation risks. Thus, in addition to the benefits to the patient, the electrodes, ICIs, electrode arrays, and systems including them and the methods for their implantation and use would not require the patient to be admitted to a hospital for observation since the intracranial complication risks are absent. An outpatient procedure with risks more comparable to a tattoo would more likely be adopted by a larger clinical and non-clinical population.

Taken together, the ICIs and ICI systems using them enable high level access to neural interfacing without the attendant risks of an intracranial penetration, making the electrode(s), assemblies and systems suitable for more widespread application due to the reduced associated health risk combined with easy and cost effective implantation.

Beyond having distinct advantages over other more invasive technologies, the ICE approach has significant advantages over non-invasive approaches such as electroencephalography (EEG), trans-cranial magnetic stimulation (TMS), and direct cortical electrical stimulation (DCES). From a recording standpoint, EEG records cortical potentials that are summed over spans of several centimeters of the surface of the cortex. Thus, the anatomic specificity of EEG would be substantially less than an intra-calvarial electrode which would be recording signals summed from only several millimeters of the cortical surface. The proximity of ICE to the cortical surface would give substantially higher cortical resolution of local electrophysiological dynamics. From cortical stimulation standpoint, this proximity would have similar advantages over what is possible with TMS and DCES. With an ICE system the close proximity to cortex in a fixed position would allow specific anatomic cortical stimulation. DCES stimulates wide regions of brain (about 5 cm) and thus cannot achieve specific cortical effects. TMS typically stimulates smaller regions but requires larger, cumbersome and quite expensive equipment that requires the patient to visit specialized centers offering access to this level of brain stimulation. An additional feature of the ICE system would be consistency of stimulation. Most cortical stimulation regimes require that a region of brain be stimulated repetitively over the course of days and weeks. As a result, TMS and DCES systems need to be affixed to the same location relative to the patient's brain on a daily basis. Because ICE systems are implanted, stimulation would be almost identical each time, while TMS and DCES are much more variable. Finally, because multiple electrodes per implant could be implanted, stimulation could be further locally tailored to optimize the localized effect. This enhanced stimulation localization may be achieved by using direct cortical stimulation methods and selecting specific stimulating electrode(s) from the multiple electrodes of an electrode assembly or electrode array, or by using frequency interference methods using selected electrode pairs from the electrode array or electrode assembly for delivering the high frequency stimulating signals. This local adjustment flexibility is not possible with TMS of DCES.

Beyond the advantages of anatomic location of the ICE system, the circuitry of the system could also be configured to perform both recording and stimulation concurrently to synergize the advantages of both modalities. Closed loop cortical stimulation has been shown to substantially improve the intended functional and physiologic effect of stimulation. Using various physiologic biomarkers taken from cortex can better inform the amplitude, timing, and stimulation regime. Such markers include, time series measures (peak and trough of select frequency rhythms) frequency band amplitudes (e.g. delta, theta, mu, alpha, beta, and gamma), connectivity measures (correlation, mutual information, etc), cross frequency interactions (e.g. phase amplitude coupling). All these signals could be used to optimize the timing and magnitude of stimulation. As an example, stimulation of memory associated areas of brain (e.g. dorsal lateral prefrontal cortex) may better improve the subject's memory by timing the stimulation with specific phases of a theta rhythm (3-5 hz). Another example would be that the magnitude or amplitude of stimulation of motor cortex for the treatment of Parkinson patients could be titrated to minimize beta-gamma phase amplitude coupling.

In addition to optimizing the functional effect, this enhancement also may provide better efficiencies in power utilization. Optimal stimulation regimes would reduce the need for continual stimulation. Additionally, if functional effects are optimized for the given current density delivered, this may allow for a reduced amount of current required for a given effect (because the closer proximity of the ICE to the brain region being stimulated). Both the optimized timing and dosage would require less power to deliver a given effect.

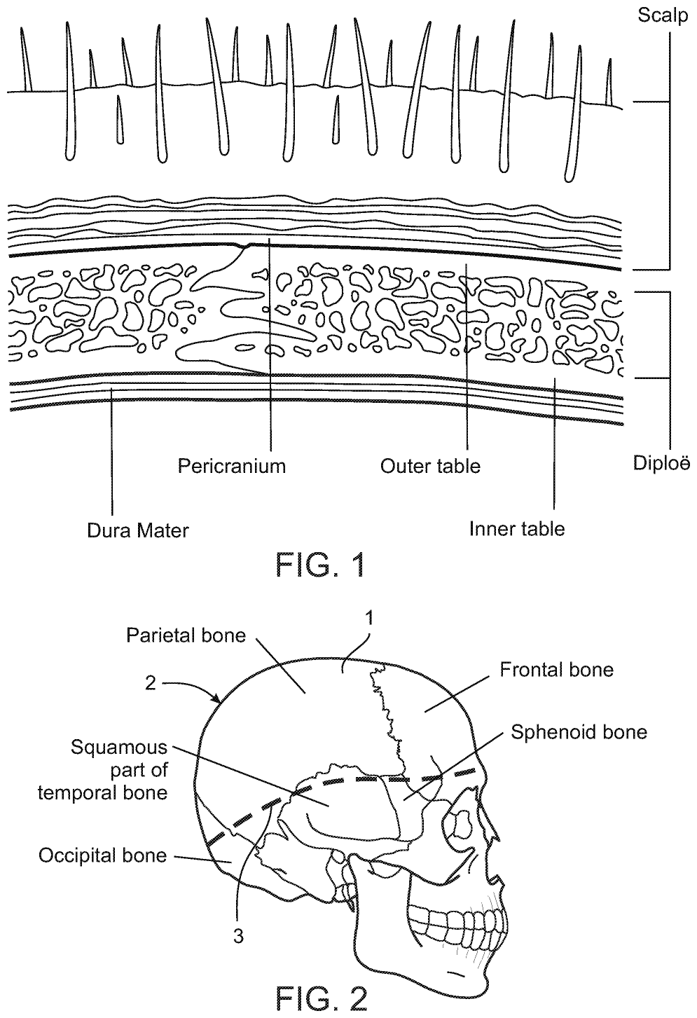

Reference is now made to FIGS. 1-2. FIG. 1 is a schematic, part cross-sectional diagram illustrating a part of a calvarial bone and the scalp. FIG. 2 is a schematic side view illustrating a human skull and some of the calvarial bones thereof.

Turning to FIG. 1, the calvaria or skullcap is the upper part of the neurocranium and covers the cranial cavity containing the brain. The calvaria is made up of the superior portions of the frontal bone, occipital bone, and parietal bones. Most bones of the calvaria include an outer table and an inner table, separated by diploe. The inner layer and the outer layer are layers of compact (and dense) bone. The diploe is a layer of cancellous (sponge-like) bone containing red bone marrow during life, through which run canals formed by diploic veins. The inner table of bone is thinner than the outer table, and in some areas there is only a thin plate of compact bone with no diploe.

Turning to FIG. 2, the positions of the frontal bone, the right sphenoid bone, the right temporal bone, the right parietal bone and the occipital bone of a human skull are indicated. The dashed line 3 indicates the approximate border between the Calvaria 1 and the lower parts of the skull. The Calvaria includes the superior portions of the frontal bone, occipital bone, and parietal bones.

The intra-calvarial electrodes penetrate the outer table and the diploe (cancellous bone layer), but not the full thickness of the inner table of the compact bone of the inner table. This would put the implanted electrode(s) in close proximity to the cortical surface. There are several methods for implanting the ICIs of the present application within the calvaria.

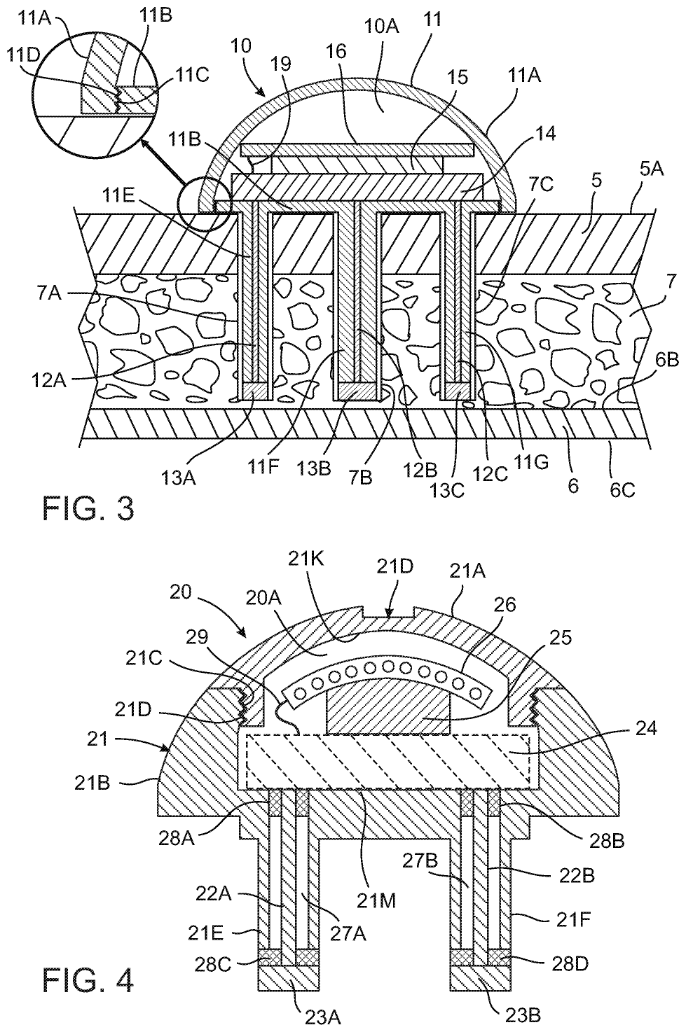

Reference is now made to FIGS. 3-4 which are schematic part cross-sectional diagrams illustrating two different intra-calvarial implants (ICI), in accordance with some embodiments of the ICIs of the present application.

Turning to FIG. 3, the ICI 10 may include an electrode supporting member 11 which may be formed in the shape of a hollow housing 11 including a dome-like hollow member 11A and a threaded base member 11B. The base member 11B has a thread 11C and the dome like hollow member 11A has a matching threaded part 11D into which the hollow member 11A may be screwed to seal the ICI 10. The base member 11B has three hollow elongated members 11E, 11F and 11G that may be formed as an integral part thereof as shown in FIG. 3, or alternatively may be detachably attached to suitable openings in the base member 11B (not shown).

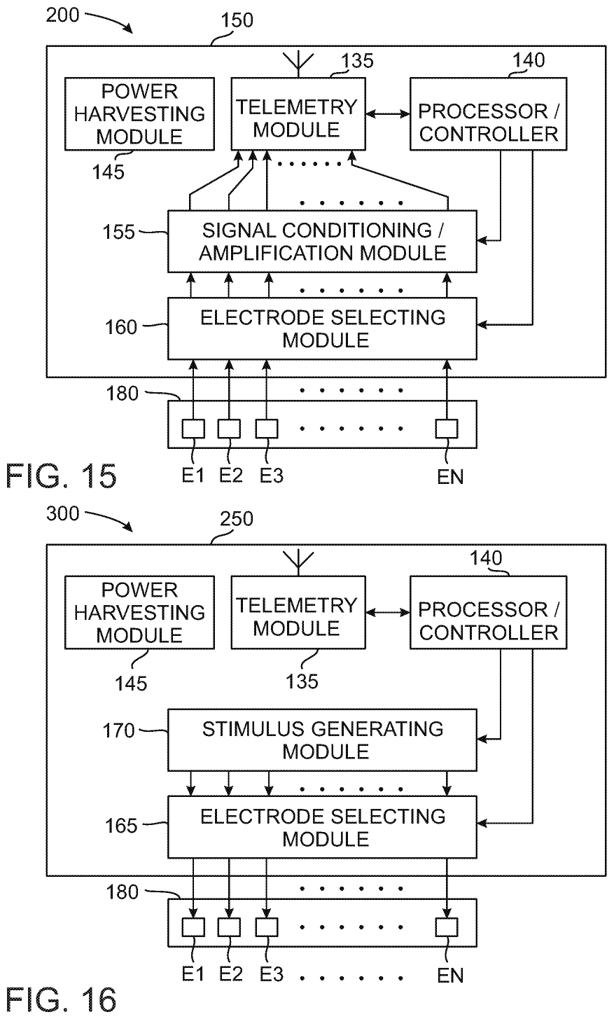

The ICI 10 may have three electrically conducting electrodes 13A, 13B and 13C suitably attached at the ends of the elongated members 11E, 11F and 11F, respectively. The ICI 10 may also include an electronic circuit module 14, an energy harvesting induction coil 16 and a spacer 15 that are disposed in the hollow space 10A enclosed within the ICI 10. The electrodes 13A, 13B and 13C are electrically coupled to the electronic circuit module 14 by three electrically conducting insulated wires 12A, 12B and 12C, respectively. The components of the electronic circuitry module are not shown in detail in FIG. 3 for the sake of clarity of illustration, but are disclosed in detail hereinafter (in FIGS. 15-18 hereinbelow). The wires 12A, 12B and 12C may conduct any electrical signals sensed by the electrodes 13A, 13B and 13C to the electronic circuit module 14 for further processing. The wires 12A, 12B and 12C may also conduct any stimulating electrical signals to any of the electrodes 13A, 13B and 13C for stimulating one or more brain regions.

The (optional) spacer 15 may be any suitable type of spacer, preferably made from an electrical insulating material (such as for example, SILGARD™ or any other polysilane or silicon based elastomer), but may be made from any type of suitable material including a metal, a polymer, a biocompatible polymer or any other suitable material.

The electrode supporting member 11 may be made from any suitably strong structural material, such as, for example, a metal, a non-magnetic metal such as titanium (which may be useful for embodiments of the ICI that need to be fMRI compatible), stainless steel, a strong polymer based material such as for example, Kevlar®, Parylene or any other suitable polymer, a synthetic ceramic material, or any other suitable material. In some embodiments, the electrode supporting member 10 or a part thereof may be coated by a layer of polymer (preferably, a biocompatible polymer such as Parylene). In some embodiments, the electrode supporting member may be shielded by a thin layer of a metallic or non-metallic, electrically conducting material such as, for example gold, platinum, iridium-platinum alloy, a metallic alloy, an electrically conducting organic material or polymer, such as for example, graphene or any other suitable electrically conducting material. The shielding coating layer may be applied using any suitable coating or plating method, such as for example, sputtering, ion beam deposition, CVD, or any other suitable method.

In some embodiments, the wires 12A, 12B and 12C, connecting the electrodes 13A, 13B and 13C to the electronic circuitry module 14 may (optionally) be shielded wires (the shielding is not shown in detail in FIG. 3 for the sake of clarity of illustration), or may even be shielded by a driven shield, depending on the particular type of electronic circuitry being used for conditioning and amplifying the sensed signals, the lengths of the wires 12A, 12B and 12C and other considerations.

The power harvesting induction coil 16 may be suitably electrically connected to the electronic circuitry module 14 by a pair of insulated electrically conducting wires 19 to provide an alternating or pulsatile current to a power harvesting module (not shown in detail in FIG. 3 but disclosed in more detail in FIGS. 15-18 hereinafter) included within the electronic circuitry module 14. The induction coil 16 may be any suitable type of electrically conducting coil suitable for harvesting energy from an externally located induction coil (not shown in FIG. 3) that is placed over or near the induct ion coil 16. It is noted that the inclusion of the induction coil 16 within the ICI 10 is contemplated only for embodiments in which the electrode supporting member is not made from a metallic electrically conducting material, and is not shielded by a coating or shielding because there may be interference with proper operation of the induction coil.

Thus, in embodiments such as the ICI 10, the electrode supporting member 11, or at least the part thereof that is disposed above or around the induction coil 16 (for example, the dome-like hollow member 11A may be made from a non-electrically conducting, non-shielded material, such as for example, Kevlar® (with or without a non-electrically conducting biocompatible coating). Thus, proper choice of the material of the electrode supporting member 11 is needed in cases where the induction coil is encased within the internal space of the electrode supporting member.

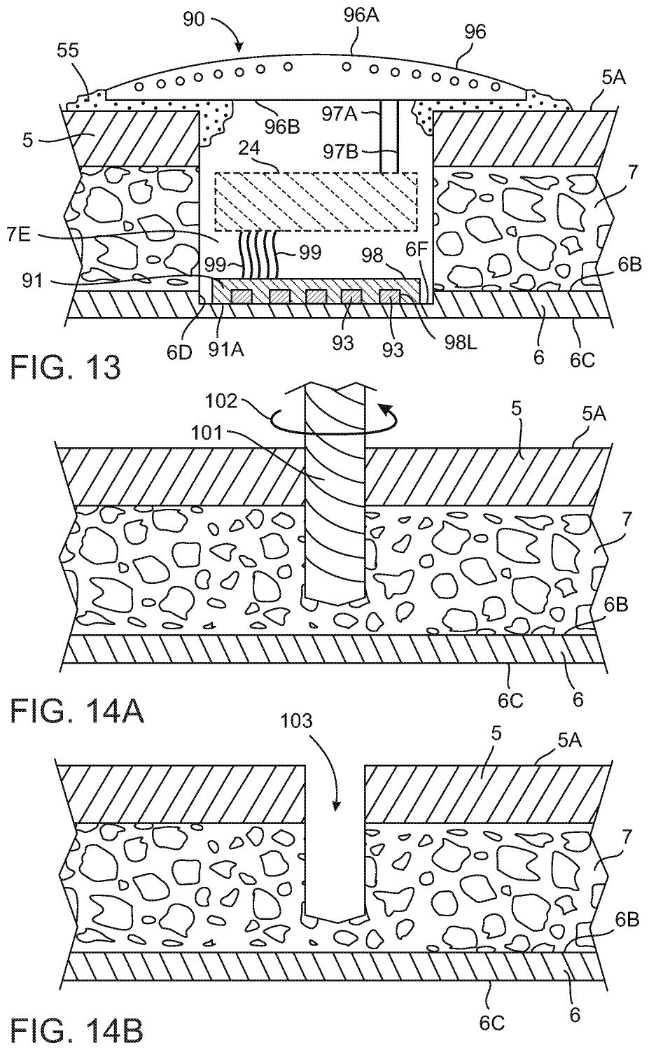

In some cases in which the induction coil is too large to be included within the electrode supporting member or in which the electrode supporting member is made from an electrically conducting (shielding) material or is coated by an electrically conducting material, the energy harvesting induction coil may be disposed outside of the electrode supporting member, such as, for example may be attached to the outer surface 5A of the outer table 5 of the calvarial bone. An example of such an embodiment is disclosed in detail in the system 90 of FIG. 13 hereinafter).

It is noted that in all embodiments of the ICIs disclosed herein in which the induction coil is disposed within (inside of) the electrode supporting member, it is assumed that the induction coil is not electrically shielded by overlying and/or surrounding parts of the electrode supporting assembly. This may be achieved by proper choice of non-electrically conducting materials material from which the electrode supporting member or of a part of the electrode supporting member which surrounds or overlies the induction coil.

It is noted that while the particular embodiment of the ICI 10 includes three electrodes (12A, 12B and 12C) this is by no means obligatory. In some embodiments of the ICIs, the ICI may include two electrodes for stimulation and/or sensing where both of the electrodes are disposed between the inner table and the outer table of the calvarial bone). For performing sensing of electrical brain signals using a differential sensing/recording arrangement.

In some other embodiments the ICI may include one electrode disposed between the inner table and the outer table of the calvarial bone and a reference electrode for performing sensing and stimulation. The reference electrode may be disposed between the inner table and the outer table of the calvarial bone or may be disposed anywhere on the housing of the ICI. The position of such a reference electrode on the housing may vary such that in some embodiments, the reference electrode is near or in contact with the outer table of the calvarial bone or the scalp of the patient. In other embodiments, the reference electrode may be attached to a separate.

Thus, the ICIs disclosed herein typically include at least one reference electrode (in some embodiments there may be more than one reference electrode) and one or more electrodes disposed between the inner table and the outer table of the calvarial bone. The reference electrode(s) may be disposed near or in contact with the outer table or even in contact with the scalp as desired.

It is noted that the term “reference” electrode throughout the present application is used when referring to use in sensing/recording. When such a “reference” electrode is used for stimulation, the same electrode may be referred to as a “return” electrode. For example, in the ICIs of the present application it is possible to have one “working” electrode or “stimulating” electrode disposed in the vicinity of the inner table and another typically (but not obligatorily) larger “return” electrode disposed in or under the scalp. During stimulation, one or more current pulses may be passed between the “working” electrode and the “return” electrode. Such a “return” electrode is also commonly referred to in the art as a “ground electrode” in a stimulation situation.

Therefore, throughout the present application, the term “reference electrode” means a reference electrode during sensing/recording and means a “return electrode” or “ground electrode” during stimulation.

In some embodiments, the ICI may include N electrodes where N is an integer number. Typically, N=1-200, however, the number of electrodes may be limited by the available cross sectional area of the part(s) of the electrode supporting member to which the electrodes are attached, the minimal electrode surface required for proper recording, mechanical and manufacturing considerations, the desired recording resolution, the distance between the electrodes and the surface of the cortex, the number of individual sensing/stimulating channels supported by the electronic circuitry module 14, the number of data channels supported by a telemetry unit (included in the electronic circuitry module, see FIGS. 15-18), the processing power of a processor included in the electronics circuitry module, see FIGS. 15-18) and other considerations. For ICIs of a type similar to the ICI 10, typically, two to ten elongated members attached to two to ten electrodes may be implemented.

Typically, the larger electrode 13C, may be used as a reference electrode during differential recording of voltage between the reference electrode 13B and any other selected electrode (such as, for example, the electrode 13A or the electrode 13C). However, during stimulation, the same electrode 13B may be used as a return electrode or ground electrode when paired with a working electrode (such as, for example the electrode 13A or 13C) and used for passing current between the selected working electrode and the return electrode 13B. It is noted that the electrode 13B may be referred to as a return electrode (or ground electrode) when stimulation is performed but may also be referred to as a reference electrode when (differential) sensing is performed.

It is noted that similar to the elongated members 11E, 11F and 11G and the electrodes 13A, 13B and 13C that are not of the same diameter and surface area, the different electrodes and/or elongated members of some embodiments of the ICIs need not be uniform in shape, size, diameter and cross (i.e., some electrodes and/or elongated members may be different than other electrodes and/or elongated members of the same ICI.

In some embodiments, all the electrodes and/or all the elongated members of the same electrode assembly may be identical in construction.

When the ICI 10 is implanted in a calvarial bone, a small incision is made in the scalp and the surface of the outer table of the calvarial bone is exposed. Then, suitable guiding holes in the form of three hollow passages 7A, 7B and 7C that pass within the outer table 5 and part (or, optionally, all) of the cancellous bone layer, may be formed in the calvarial bone by using a surgical drill or any other suitable type of laser based or ultrasonic or mechanical bone boring device, as is disclosed in detail hereinafter. The passages 7A, 7B and 7C penetrate the outer table and the cancellous bone 7. The elongated members 11E, 11F and 11G may then be inserted into the passages 7A, 7B and 7C, respectively until the base member 11B makes contact with the surface inner surface 6B of the inner table 6. As the elongated members 11E, 11F and 11G are orthogonal to the base member 11B, the passages 7A, 7B and 7C are drilled parallel to each other. The passages may have a diameter slightly larger than the diameter of the elongated member to allow easy insertion of the elongated members 11E, 11F and 11G into the hollow passages 7A, 7B and 7C. For a specific electrode configuration, a suitable drilling template may be used with holes made in the template (such as, for example a suitably perforated stainless steel plate) matching the desired passage diameter and inter electrode distances.

In some embodiments the base plate 11B and/or the upper part of the hollow passages and/or the part of the hollow member 11A may be sealed by the application of a suitable (preferably biocompatible) glue or sealant. For example, dental acrylic may be used. The glue or sealant may attach the base member 11B to the outer surface 5A of the outer table 5 to form a complete seal of all the hollow passages 7A, 7B and 7C.

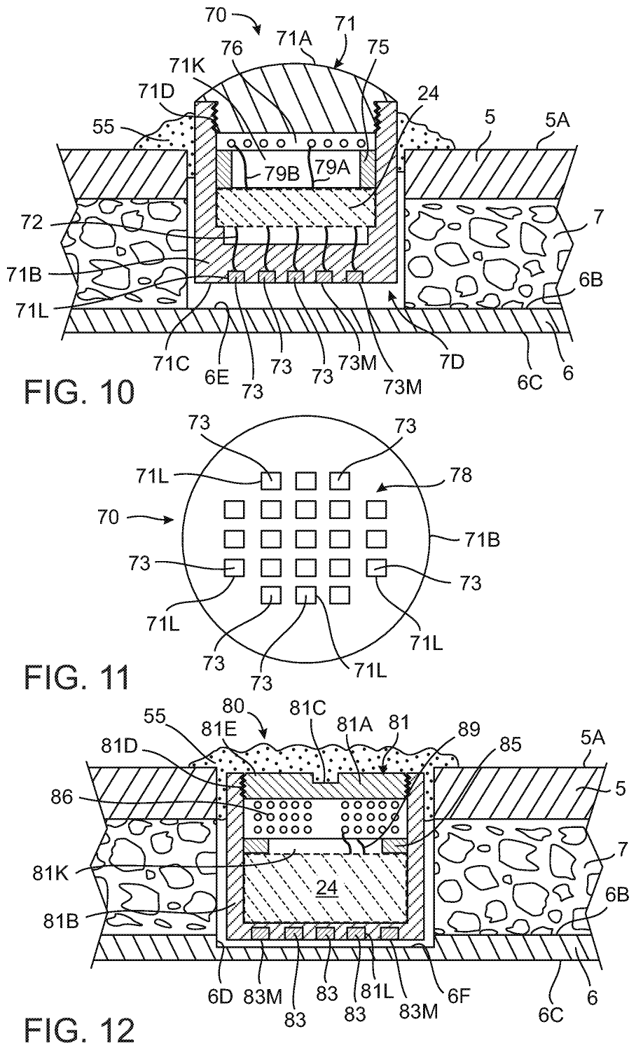

In some embodiments, three to five electrodes placed approximately 2-3 mm apart may be used. This could allow for recording from a focal region and may also allow stimulation across electrodes that could be “steered” by varying the current across the electrodes to achieve an optimal field density in the regions beneath the ICI (or electrode array if used, see, for example, FIGS. 10-11 and 13 hereinafter) to optimize the desired neural effect.

Turning to FIG. 4, the ICI 20 includes a hollow electrode supporting member 21, two hollow elongated members 21E and 21F are formed as an integrated part of the base member 21B. Alternatively, in some embodiments, the hollow elongated members may be separate parts that may be suitably attached to or screwed into the base member 21B by using suitable threads and threaded holes in the base member 21B (holes and threads are not shown in FIG. 4).

The electrode supporting member 21 is a hollow member having a hollow space 20A therein. The electrode supporting member 21 also includes a lid 21A that has a threaded part 21C. The base member 21B may be made from titanium, and the lid 21A may be made from an electrically non-conducting material such as, for example Kevlar®. The lid 21A may be screwed into a matching threaded opening 21D formed in the base member 21. The ICI 20 also includes an electronic circuitry module 24, a curved power harvesting coil 26 and a spacer 25 interposed between the electronic circuitry module 24 and the coil 26.

The power harvesting induction coil 26 may be suitably electrically connected to the electronic circuitry module 24 by a pair of insulated electrically conducting wires 29. Two electrodes 23A and 23B are suitably attached to the ends of the two elongated members 21E and 21F, respectively. The electrodes 23A and 23B are electrically isolated from the elongated members 21E and 21F by annular members 28C and 28D which may be made from a non-electrically conducting material such as for example Parylene. Two additional annular members 28A and 28B are disposed within the hollow spaces 27A and 27B close to where the passages 27A and 27B open at the upper surface 21M of the base member 21B, The annular members 28A and 28B may also be made from a non-electrically conducting material such as, for example, Parylene. Two electrically conducting insulated wires 22A and 22B may electrically connected to the electrodes 23A and 23B, respectively, and to the electronic circuitry module 24.

The curved induction coil 26 may be parabolically shaped but may also be of a different curved shape. In accordance with some embodiments, the induction coil 26 may be shaped to fit the inner curved concave surface 21K of the lid 21, and the induction coil 26 may also (optionally) be attached to the surface 21K (by suitably increasing the height of the spacer 25 or by eliminating the (optional) spacer member 25 altogether. The implantation of the ICI 20 may be performed in a similar way to the method described for the ICI 10, with the exception that two hollow passages in the calvarial bone (instead of the three passages required for the implantation of the ICI 10) may be required for implantation.

It is noted that in accordance with some embodiment in which the elongated members are orthogonal to the lower surface of the base member of the electrode supporting member, the hollow elongated members may be made from a stiff and strong material (such as, for example, titanium) and the electrodes may be shaped to have sharp penetrating tips (not shown) For example, the electrodes may be shaped like cones having their tips directed towards the outer surface 5A of the outer table 5 during implantation. In such exemplary embodiments, no hollow passages would be required prior to implantation. In this configuration the electrodes may have sufficient structural strength and penetrability that the entire ICI may be hammered or punched into the skull and the electrodes and the elongated member may make their own hollow passages by forcefully penetrating into the outer table 5 and the cancellous bone 7 without penetrating the full thickness of the inner table 6.

It is noted that the orthogonality of the elongated members of the ICIs 10 and 20 to the respective base members is not obligatory.

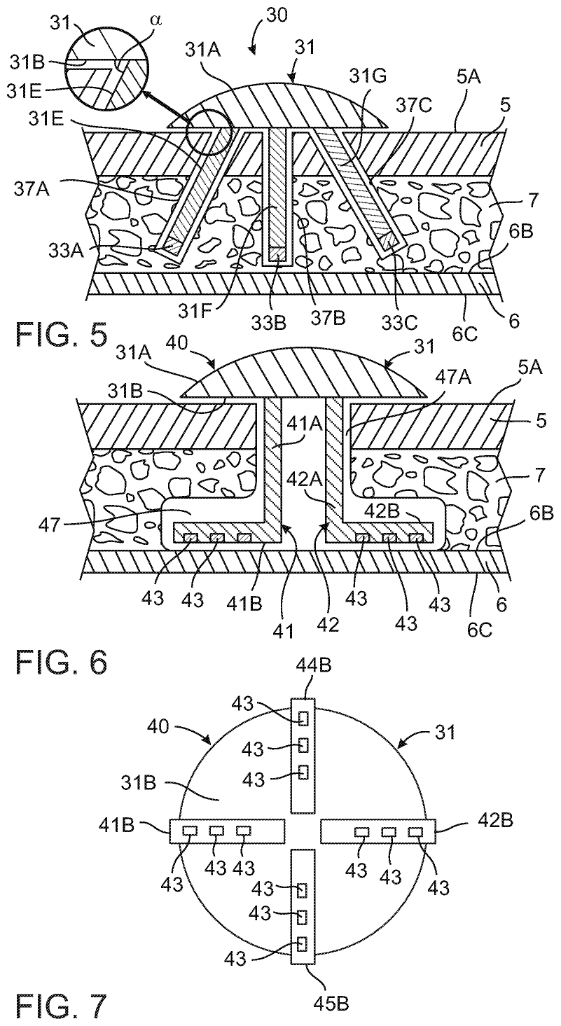

Reference is now made to FIG. 5 which is a schematic part cross-sectional view, illustrating an ICI having obliquely arranged elongated members carrying electrodes. It is noted that for the sake of clarity of illustration, the structure of the components of the ICI 30 is not shown in detail and only the details illustrating the relation of the elongated members supporting the electrodes to the electrode supporting member are shown. It may be assumed that the structure and function of such components may be similar to those of the ICIs 10 and 20 as disclosed in detail hereinabove. The ICI 30 includes an electrode supporting member 31 that has a curved surface 31A and a flat (planar) surface 31B. The ICI 31 has three elongated members 31E, 31F and 31G. Each of the elongated members 31E, 31F and 31G has an electrode 33A, 33B and 33B attached to an end thereof, respectively. The three elongated members may be arranged relative to the surface 31B, such that the elongated member 31F may be orthogonal to the surface 31B, while each of the elongated members 31E and 31G may be inclined at an angle α to the plane of the surface 31B. Typically, a is in the range of 15-75°, but angles higher or lower than this range may also be implemented.

However, in accordance with some other embodiments, the three elongated members may also be equally spaced apart from each other so the distance between each pair of electrodes may be similar (not shown in FIG. 5). Additionally, each of the elongated members 31E, 31F and 31G may be obliquely attached the to the surface 21B (or may be formed as an integral part of the electrode supporting member 31) such that the angles α formed by each elongated member to the planar surface 31B may be identical (the angle α may vary in the range of 15-75°, but angles higher or lower than this range may also be implemented). In some embodiments (not shown), the angles implemented between each of the elongated members may not be identical (for example, one elongated member may be inclined at an angle of 30° to the surface 31B while another elongated member may be inclines to the surface 31B at an angle of 45°). In such cases, the length of the elongated members may be different from the length of the other elongated members to keep the distance from the electrodes 33A, 33B and 33C to the inner surface 6B of the inner table 6 roughly the same for all electrodes.

In some other embodiments, there may be two electrodes attached to two elongated members or there may be more than three electrodes and more than three elongated members and each of these may be inclined at a different angle relative to the surface 31B. In some embodiments, some electrodes may be orthogonal, while others may be angled. All combinations and permutations of the number of elongated members, angles of inclination of the elongated members relative to the surface 31B and electrode lengths may be implemented and are contemplated to be included as possible embodiments of the ICIs of the present application.

The implantation of such ICIs may be similar to the implantation methods disclosed hereinabove with two main exceptions. The first is that some of the hollow passages (such as the hollow passages 37A, 37B of FIG. 5) may have to be drilled at an angle to the upper surface 5A of the outer table 5. The second is that in accordance with some embodiments, at least some (or optionally all) of the elongated members (such as, for example, the elongated members 31E and 31G, and/or also the orthogonal elongated member 31F) may have to be made from a flexible or elastic material in order to allow bending and flexing of the elongated member to make the insertion into the angled hollow passages (for example, the hollow passages 37A and 37C) easier and more convenient. Such flexible/bendable/elastic materials may include, for example any type of biocompatible elastomers such as polysilans, polyurethans, thermoplastic polyurethans, polysiloxane modified styrene-ethylene/butylene block copolymers, or any other type of suitable flexible or elastic biocompatible material.

The above oblique arrangement types of ICIs (such as for example, the ICI 30) may have several advantages. First, the electrode may span a wider area due to the increased length and inclination angles of some of the elongated members. Since the electrodes would be placed from a central location, the amount of skin (scalp) required to be cut during implantation may be reduced. Both these approaches may require standard drilling techniques in which a standard surgical drill is used to make holes in the skull. In addition to the standard drill, temporary templates affixed to the skull could be used that control both depth and trajectory of the drill bit. Such templates may be shaped to ensure that the hollow passages in the skull are in the correct depth, angle and spacing relative to other drill holes. Beyond standard drill applications, one could also form hollow passages in the skull using a number of other different methods. These methods may include, for example, microscale high intensity lasers, ultrasound bone cutters, or stereotactic screws to name a few.

It is noted that while the elongate members of the embodiments disclosed hereinabove were straight elongated members, this is not obligatory for practicing the ICIs of the present application.

Reference is now made to FIGS. 6 and 7. FIG. 6 is a schematic cross sectional view illustrating an ICI having bent elongated members including multiple electrodes, in accordance with some embodiments of the ICIs of the present application. FIG. 7 is a schematic bottom view of the ICI of FIG. 6.

It is noted that the ICI 40 is note shown in full structural detail and some of the components thereof are shown only in sufficient details for demonstrating the differences relative to other embodiments of the ICIs disclosed hereinabove. The ICI 40 may include the electrode supporting member 30 as disclosed hereinabove and four bent (L-shaped) electrode carrying members 41, 42, 44 and 45. The electrode carrying members each include three electrodes 43 (it is noted that the electrode carrying members 44 and 45 are not illustrated in the cross sectional view of FIG. 6 and are best seen in FIG. 7. Each electrode carrying member includes a first part (for example, the first parts 41A and 41B of the electrode carrying members 41 and 42, respectively, that may be orthogonal to the surface 31B and a second part (for example, the second parts 41B and 42B. The second part 41B is bent at approximately 90° relative to the first Part 41A and the second part 42B is bent at approximately 90° relative to the first Part 42A. Each of the second parts 41B, 42B, 44B and 45B includes three electrodes 43.

The electrodes 43 may be embedded within or attached to the second parts 41B, 42B, 44B and 45B and when the ICI 40 is implanted the electrodes 43 may be disposed close to or in contact with the inner surface 6B of the inner table 6. The electrodes 43 are electrically connected to an electronic circuitry module (not shown in detail but may be identical to the electronic circuitry module 14 of the ICI 10) housed within the electrode supporting member 31, as disclosed hereinabove by suitable electrically conducting insulated wires (not shown) running within the electrode carrying members 41, 42, 44 and 45. As seen in FIG. 7, may be equally spaced from each other and the second parts 41B and 42B may lie in a direction which is orthogonal to a line along which the second parts 44B and 45B may lie.

It is noted that the electrode carrying members 41, 42, 44 and 45 may be disposed within an internal cavity or chamber 47 that is formed within the cancellous bone 7 of the calvaria or skull bone, as seen in FIG. 6, A single entry hole or passage 47A that opens in the outer table 5 may be expanded or enlarged laterally with respect to the entry passage 47 to form a laterally extending cavity or chamber 47 within which the electrodes 43 are disposed (preferably, close to or in contact with the surface 6B of the inner table 6).

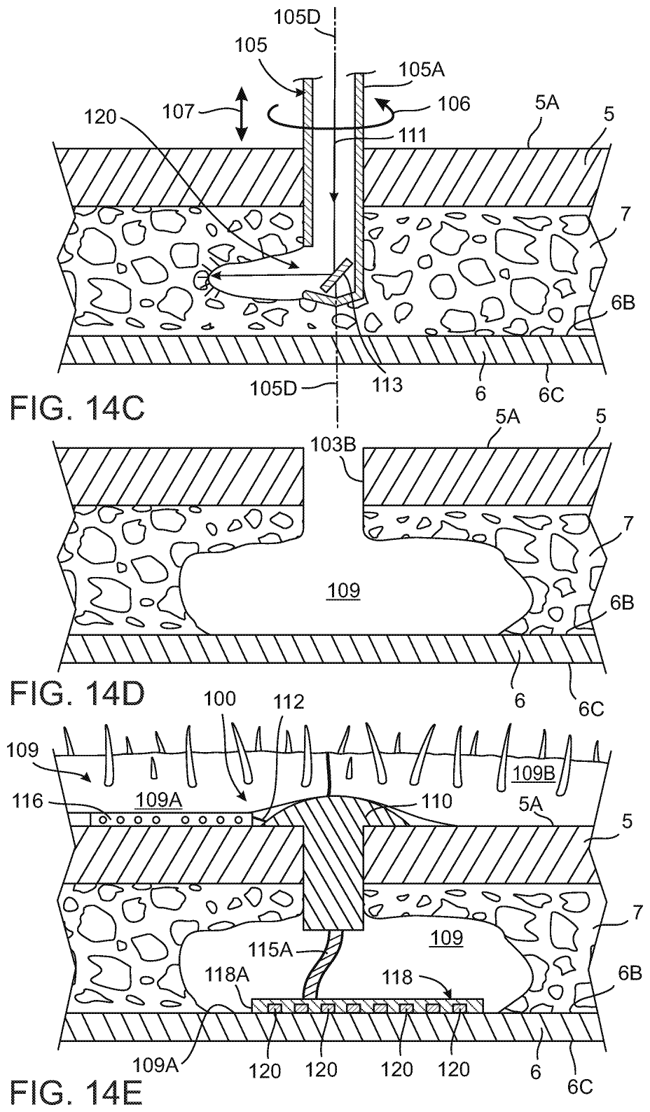

Alternatively, a single-entry burr hole that is orthogonal to skull is formed in the outer table 5 (and possibly within a part of the cancellous bone 7) and an additional four tunnels or hollow passages (not shown) may then be formed at right angles to the entry burr hole. Such tunnels that may be parallel to the inner surface 6C of the inner table 6 may radiate out from the central hollow passage 47A. The advantage of such a configuration would be that the entry site (or burr hole) may be rather small and therefore be minimally invasive and more electrodes may be placed that cover a larger area of the inner table 6 and therefore may be used to sense/record and or stimulate a larger cortical region with a higher resolution in sensing/recording and higher current density in stimulation. The drilling for implanting the ICI may require more advanced drilling and bone sculpting techniques. Using drills, a central pilot hole may be drilled and then small right angle drills may be used to form the radially extending electrode tunnels or the chamber 47A. Alternatively one may use high energy lasers (femto lasers) for bone sculpting. Exemplary methods are disclosed in detail herein after with respect to FIGS. 14A-14E.

It is noted that, the ICIs disclosed in the present application is not limited to the configurations of the specific embodiments disclosed hereinabove. Rather, some other embodiments may include electrode carrying members that are able to extend intra-calvarially using extended curved trajectories of the electrode carrying member(s) that may pass across longer stretches of skull to cover multiple anatomic regions in the brain.

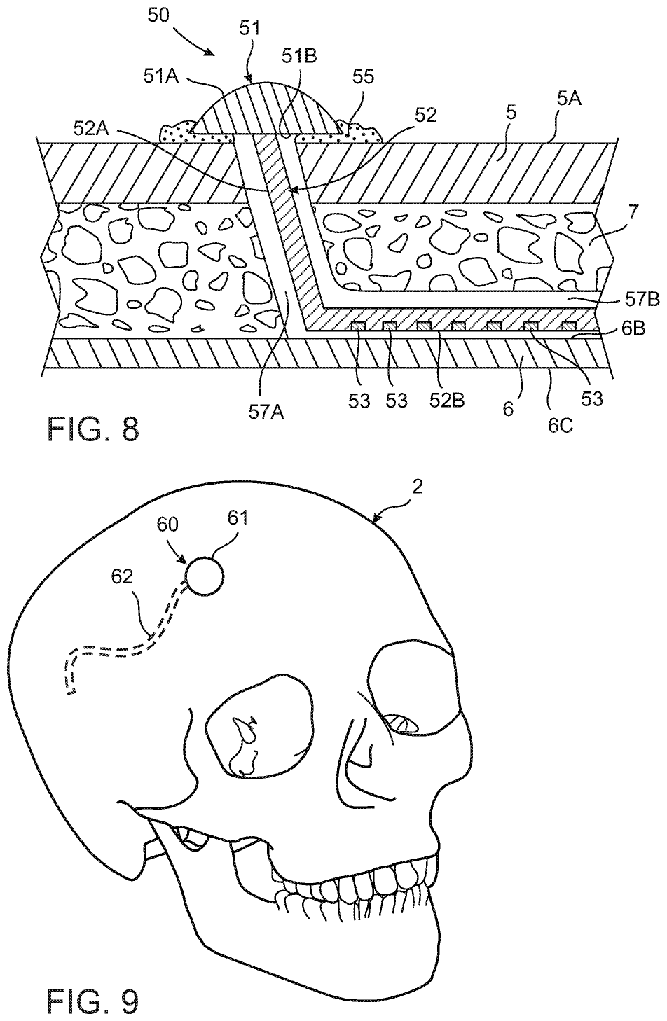

Reference is now made to FIGS. 8 and 9. FIG. 8 is a schematic part cross-sectional view illustrating an ICI having a single extended linear electrode carrying member having multiple electrodes therein, in accordance with some embodiments of the ICIs of the present application. FIG. 9 is a schematic isometric view illustrating a skull with an implanted ICI having an extended flexible electrode carrying member with multiple electrode extending intra-cranially within the calvaria, in accordance with some embodiments of the ICIs of the present application.

The ICI 50 may include an electrode supporting member 51 having a curved surface 51A and a flat (planar) surface 51B. After implantation, the surface 51B may be sealingly glued or sealed to the underlying surface 5A of the outer table 5, by using any type of (elastomeric or non-elastomeric) sealant material 55 as disclosed in detail hereinabove The ICI 50 may also include an elongated electrode carrying member 52 having multiple electrodes 53, formed therein or attached thereto. The electrode carrying member 52 may be connected to the electrode supporting member 51 at the surface 51B thereof. The various internal components of the electrode supporting member 51 are not shown in detail in FIG. 8 but the structure and components of the electrode supporting member 51 may be similar to the structure and components of the electrode supporting members disclosed hereinabove.

The electrode carrying member 52 has a first part 52A (attached to the surface 51B of the electrode supporting member 51) and a second part 52B that includes multiple electrodes 53. In accordance with some embodiment, either the first part 52A or the second part 52B or both the first part 52A and the second part 52B may be made from a flexible and/or bendable and/or elastic material or structure to assist the insertion of the electrode carrying member 52 through the extended contiguous hollow passages 57A and 57B that are formed within the cancellous bone 7. For example, the electrode carrying member 52 may be made from or at least partially made from any of the elastic and/or flexible materials disclosed in detail hereinabove.

Alternatively and/or additionally, the member 52 may be made from alternating portions of flexible and rigid materials (not shown) resulting in an elongated member having the capability to bend and/or follow a tortuous path within an extended hollow tunnel or passage such as the passages 57A and 57B. For example, the electrode carrying member may be configured as a hollow cylindrical structure made from alternating annular segments that are sealingly attached to each other (not shown) wherein some of the hollow annular segments are made from or include an electrically conducting material such as, for example, titanium, gold, platinum or any other suitable electrically conducting rigid material and the other annular segments may be made from an electrically insulating (non-electrically conducting) material(s) that are elastic or flexible), the flexible annular segments may confer flexibility and bending capability on the entire composite segmented structure of such an electrode carrying member while the central hollow passage formed by the serially attached annular segments may be used to enclose therein the insulated wires connecting each of the electrodes to an electronic circuitry module (not shown) that may be included in the electrode supporting member 51.

There are several methods for making elongated flexible multi electrode members that may be used in the ICIs of the present application and placed in an intracalvarial location. For example, a variety of medical flexible electrode catheters are currently commercially available. The technology common to all of these catheter designs is the application of one or more metallic bands on a catheter body. Examples of medical catheters using metallic banded electrodes include permanent and temporary cardiac pacing leads, electro-physiologic (EP) catheters, electrocautery probes and spinal stimulation catheters. The use of pre-formed metallic band electrodes manufactured from noble metals, such as gold or platinum and various other conductive alloys has found widespread application.

Metallic band electrodes possess distinct steerability problems. The steerability problems arise from the inflexible nature of the metallic circular rings or bands. These inflexible bands of metal are typically adhesively bonded, crimped or otherwise attached to the exterior surface of the catheter or lead body. The bands are electrically coupled to electrical conductors that typically extend through one or more lumens in the catheter or lead body. The bands tend to be relatively thick and are therefore rigid. For neurological applications, the bands are typically about 3 millimeters wide. When it is considered that four or eight such ring electrodes are typically spaced about four millimeters apart along the distal end portion of the catheter body, they can impact the ability of the distal end portion of the catheter or lead to flex and conform to channel structures.

As noted above, band electrodes placed on a flexible catheter or lead stiffen the catheter or lead and thereby reduce steerability. The steerability may be enhanced by having at least one slot cut through the electrically conductive band to confer flexibility. The band electrode may include electrically conductive bands having an outside diameter in the range of 1-4 mm. The conductive band may be an electrically conducting biocompatible material such as, for example, platinum, gold, silver, platinum-iridium, stainless steel and MP35N® alloy or may be any other suitable biocompatible alloy of metals. The lead for a medical device includes the band electrodes as described above and may further include a lead body having one or more electrically conducting electrically insulated thin wires extending from a proximal end and a distal end of the lead body. Each wire may be connected at one end to a band electrode. The lead body may have an outside diameter in the range of 1-4 mm. The electrode and lead may be made by a method comprising providing a conductive cylindrical band and cutting at least one slot through the band to confer flexibility. Furthermore, a hollow lumen may be included in the multi-electrode lead into which lumen pre-shaped stylets may be inserted that may help guide the multi-electrode lead through the pre-configured channels in the cancellous bone.

U.S. Pat. No. 6,493,590 to Wessman et al., discloses flexible multi-band electrodes for medical leads which have improved flexibility. Some of the leads and electrodes disclosed by Wessman et al. may also be used, in any of the ICIs requiring such flexible multi-electrode arrays. Such ICIs may include, for example, the ICIs 40, 50, 60, 61 and 100.

In an Article by Patrick J. Rousche et al. entitled “Flexible Polyimide-Based Intracortical Electrode Arrays with Bioactive Capability” published in IEEE TRANSACTIONS ON BIOMEDICAL ENGINEERING, VOL. 48, NO. 3, March 2001 361, the authors disclose a method for making elongated flexible multi electrode members is by photo-lithography. In this method, a thin-film, polyimide-based, multichannel intracortical Bio-MEMS interface can be manufactured with standard planar photo-lithographic CMOS-compatible techniques on 4-in silicon wafers. The use of polyimide provides a mechanically flexible substrate which can be manipulated into unique three-dimensional designs. Polyimide also provides a good surface for the selective attachment of various important bioactive species onto the device in order to encourage favorable long-term reactions at the tissue-electrode interface. Structures have an integrated polyimide cable providing efficient contact points for a high-density connector. The ICIs of the present application may include such flexible Polyimide-based electrode arrays for placement within channels formed within the calvarial bone. Such ICIs may include, for example, the ICIs 40, 50, 60, 61 and 100.

Thus, it is possible to use such embodiments including flexible/bendable electrode carrying members to be implanted in a complex curved hollow passage within the cancellous bone 7, such a passage may have a trajectory that follows a specific functional region or network of such regions over the brain. As an example, a single entry point on the vertex of the skull may be formed and a hollow passage (electrode tunnel tract) may be made formed in the cancellous bone that passes over the full length of motor cortex with multiple electrodes included in the electrode carrying member disposed within (such as for example the electrode carrying members 52, of FIG. 8.) the full length of this electrode carrying member).

This configuration may enable various types of stimulation and recording. For example, in an exemplary, non-limiting brain computer interface (BCI) applications one could record from the arm, hand, and face areas of the cortex. Comparable levels of cortical coverage using conventional prior art Ecog implantation methods would require a larger open surgery with the attendant risks. Implantation would require advanced bone sculpting techniques to manage the formation of tortuous hollow passages and curvatures. Specifically, one would use a steerable catheter or endoscopic device with a fiber optic cable and sapphire tip that is capable of emitting high energy discharges for bone sculpting/penetration. Furthermore, such an embodiment may require co-registration with a stereotactic navigation system, using CT, MRI or other imaging techniques. This may allow for safe movement of the electrode carrying member through extended regions of the skull. Also, with co-registration with anatomic and functional MRI (fMRI) imaging, electrodes can be optimally placed within the skull to overly intended targets brain regions.

Turning briefly to FIG. 9, the ICI 60 may include a housing 61 (for housing the electronic circuitry module which is not shown in FIG. 9 for the sake of clarity of illustration) attached to an elongated flexible or bendable electrode carrying member 62. The electrode supporting member 62 may be similar to the electrode carrying member 52 of FIG. 8, but may be any other type of suitable flexible/segmented/bendable electrode carrying members as disclosed herein. For example, the flexible electrode carrying member 62 may be a thin elongated lead-like or catheter-like member having multiple electrodes attached thereto or integrated therein. In some embodiments a reference electrode (not shown in FIG. 9) may be attached to the housing 61 and differential recording may be performed between any selected electrodes and/or electrode groups of the elongated flexible electrode carrying member 62 and the reference electrode attached to the housing. However differential recording may also be performed by any two selected electrodes of the electrode supporting member 62.

For electrical stimulation of the brain tissue underlying the electrodes of electrode supporting member 62, current pulses may be passed between any selected pair(s) of the multiple electrodes of the electrode supporting member 62, or between one or more of the electrodes of electrode supporting member 62 and the reference electrode (not shown) of the housing 61.

Reference is now made to FIGS. 10-12. FIG. 10 is a schematic, part cross-sectional view of a “monoblock type” ICI having an integrated electrode array and implanted within a calvarial bone, in accordance with some embodiments of the ICIs of the present application. FIG. 11 is a schematic bottom view of the ICI of FIG. 10. FIG. 12 is a schematic, part cross-sectional view of another “monoblock type” ICI having an integrated electrode array disposed within a recess made in the inner table of a calvarial bone, in accordance with some embodiments of the ICIs of the present application.

Turning to FIGS. 10-11, the ICI 70 may include a hollow cylindrical housing 71 having a cylindrical base member 71B and a lid 71A. In some embodiments, the base member 71B and the lid 71A may be made from a strong electrically insulating material such as, for example, Kevlar® or any other suitable material and may be coated by a layer of biocompatible material such as, for example, Parylene®. The lid 71A has a threaded portion 71D that may be tightly screwed into a matched female thread formed in the upper open end of the base member 71B to hermetically seal the housing 71. The ICI 70 may also include the electronic circuitry module 24, an annular spacer 75 and an induction coil 76 disposed within the hollow space 71K formed within the housing 71. The annular spacer 75 may separate between the electronic circuitry module 24 and the induction coil 76.

The induction coil 76 is suitably electrically coupled to the electronic circuitry module 24 by a pair of electrically conducting insulated wires 79A and 79B. The portion of the base member 71B facing the surface 6E of the inner table 6 includes twenty one electrodes 73 that are attached within suitable twenty one recesses 71L formed in the surface 71C of the base member 71B. The electrodes 73 may be formed from any suitable electrically conducting material as disclosed in detail hereinabove. The electrodes 73 may be attached within the recesses 71L such that the surfaces 73M of the electrodes 73 are flush with the surface 71C of the base member 71B. Alternatively, in some embodiments, the surfaces 73M of the electrodes 73 may protrude from the surface 71C. Each of the electrodes 73 is suitably electrically connected to the electronic circuitry module 24 by an insulated electrically conducting wire 72.

This type of ICI is referred to hereinafter as a “monoblock” ICI. The arrangement of the electrodes 73 on the surface 71C of the mono-block ICI 70 forms an electrode array 78 (best seen in FIG. 11) that is integrated into the base member 71B. The advantage of such a mono-block configuration is that it allows the shortening of the distance between the electrodes 73 and the electronic circuitry module 24 with concomitant shortening of the length of the wires 72 which may improve the SNR.

When the monoblock ICI 70 is implanted in a skull, the implantation method may include making an incision in the scalp (not shown in FIG. 10) overlying the outer table 5 of the calvarial bone (or any other skull bone) to expose the surface of the bone, forming a cylindrical hollow passage 7D passing through the outer table 5 and some or all of the cancellous bone 7. In accordance with some embodiments, the passage 7D may end in a flat (planar) surface 6E that may be formed by using a suitable milling tool (not shown or a suitable abrasive coated flat surfaced tool (not shown in FIG. 10) inserted into the passage 7D and rotated to flatten and/or polish the outer surface 6B of the inner table 6. After the passage 7D is formed, the ICI 70 may be inserted into the passage 7D until the electrodes 73 of the electrode array 78 are in contact with or in close proximity to the flattened surface 6E.

Optionally, the entire ICI may be rotated while testing the sensing/recording and/or stimulating of the ICI 70 to optimize the positioning and orientation of the electrodes 73 (which may be required in embodiments in which the electrodes 73 are arranged in an elongated type of array configuration). A suitable biocompatible sealant 55 may then be used to seal the opening of the passage 7D, as illustrated in FIG. 10. After the sealant 55 sets and/or hardens, the skin flaps of the incision made in the scalp (not shown) may be closed to allow the scalp to cover the implantation region and the assembly and to allow the scalp to heal.

It is noted that the number of the electrodes 73 illustrated in FIGS. 10-11 is not limiting and any number of electrodes having any size and/or shapes may be used to form the electrode array 78. Typically, the number of the electrodes in the array may be in the range of 2-200 electrodes. However, the number of electrodes may be affected by the total area of the surface 71C, the minimal acceptable electrode size and/or surface area required for sensing and/or stimulation, the specific application and other practical engineering and manufacturing methods and considerations.

Turning to FIG. 12, the ICI 80 may include a hollow cylindrical housing 81 having a cylindrical base member 81B and a flat lid 81A. In some embodiments, the base member 81B and the lid 81A may be made from a strong electrically insulating material such as for example, Kevlar® or any other suitable material and may be coated by a thin layer of biocompatible material such as, for example, Parylene®. The lid 81A has a threaded portion 81D that may be tightly screwed into a matched female thread formed in the upper open end of the base member 81B to hermetically seal the housing 81.

The lid 81A may have a suitable notch or recess 81C formed therein that may be used for inserting a tool (such as, for example, a screwdriver) into the lid, for screwing the lid 81A, in or out of the housing 81 during manufacturing and/or assembling of the ICI 80. The ICI 80 may also include the electronic circuitry module 24, an annular spacer 85 and an induction coil 86 disposed within the hollow space 81K formed within the housing 81. The induction coil 86 useable for power harvesting may be a multilayer/multi-winding type of coil as illustrated in FIG. 11, but in some embodiments, a single layered induction coil (planar or curved, as disclosed in detail hereinabove) may be used. The induction coil 86 is suitably electrically coupled to the electronic circuitry module 24 by a pair of electrically conducting insulated wires 89. The portion of the base member 81B facing the surface 6C of the inner table 6 includes multiple electrodes 83 that may be attached within suitable recesses 81L formed in the surface 81C of the base member 81B. The electrodes 83 may be formed from any suitable electrically conducting material as disclosed in detail hereinabove. The electrodes 83 may be attached within the recesses 81L such that the surfaces 83M of the electrodes 83 are flush with the surface 81C of the base member 81B. Alternatively, in some embodiments, the surfaces 83M of the electrodes 83 may protrude from the surface 81C. Each of the electrodes 83 may be suitably electrically connected to the electronic circuitry module 24 by an insulated electrically conducting wire (the wires are not shown in FIG. 12 for the sake of clarity of illustration).

The ICI 80 is also a “monoblock” ICI. The arrangement of the electrodes 83 on the surface 81C of the mono-block ICI 80 forms an electrode array that is integrated into the base member 81B. The advantage of such a mono-block configuration is that it allows shortening the distance between the electrodes 83 and the electronic circuitry module 24 with concomitant shortening of the length of the wires which may improve the SNR as disclosed hereinabove in detail for the ICI 70.

When the monoblock ICI 80 is implanted in a skull, the implantation method may include making an incision in the scalp overlying the outer table 5 of the calvarial bone (or any other skull bone) to expose the surface 5A of the inner table 5 of the bone, forming a cylindrical hollow passage 7E passing through the outer table 5 and all of the cancellous bone 7. In accordance with some embodiments, the passage 7E may include a recess 6D that is formed within the inner table 6 in order to reduce the thickness of the dense bone of the inner table 6 within the recess 6D. The surface 6F at the bottom of the recess 6D may be a flat (planar) surface that may be formed by using a suitable milling tool (not shown) or a suitable abrasive coated flat surfaced tool (not shown in FIG. 12) inserted into the passage 7E and rotated to form the cavity 6D and to flatten and/or polish the surface 6F formed at the bottom of the recess 6D of the inner table 6. However, in some embodiments, the surface 6F may be a non-planar surface, such as, for example, a convex surface a concave surface or any other type of non-planar surface.

After the passage 7E is formed, the ICI 80 may be inserted into the passage 7E until the electrodes 83 are in contact with or in close proximity to the surface 6F. Optionally, the entire ICI 80 may be rotated while testing the sensing/recording and/or stimulating of the ICI 80 to optimize the positioning and orientation of the electrodes 83 for adjusting the effects of stimulation and/or sensing/recording (which may be necessary, in embodiments in which the electrodes 83 are arranged in an elongated type of array configuration). A suitable biocompatible sealant 55 may then be used to seal the opening of the passage 7E, as illustrated in FIG. 12. After the sealant 55 sets and/or hardens, the skin flaps (not shown) of the incision (not shown) made in the scalp (not shown) are closed to allow the scalp to cover the implantation region and the assembly and to allow the scalp to heal.

It is noted that after implantation, the upper surface 81E of the ICI 81 is below the level of the surface 5A of the outer table 5, such that after the sealant 55 is applied, the electronic circuitry module 24 and the electrodes 83 are all disposed within the cancellous bone layer 7 and between the outer table 5 and the inner table 6. This advantageously reduces the level of noise and increases the SNR. Additionally, the reduction of the thickness of the inner table 6 within the recess 6D further reduces the distance between the electrodes 83 and the cortical surface and reduces the impedance of the remaining dense bone of the inner table 6 that is interposed between the electrodes 83 and the surface of the cortex. These reductions in distance and bone thickness may advantageously and significantly improve the sensed signal's amplitude and reduces the SNR.

However, while in all the embodiments of the ICIs of the present application all the electrodes and all the electrode array are disposed in a cavity or chamber or tunnel or passage that is formed (and is disposed) between the outer table 5 and the inner table 6 of the calvarial bone (or the skull bone), the electronic circuitry modules being used and/or the induction coils being used may be disposed in other different locations. For example, in the ICIs 10, 30 and 50, the electronic circuitry modules and the induction coils that are included within the electrode supporting members 11, 31 and 51, respectively that are disposed outside of the surface 5A of the outer table 5 and are therefore not disposed between the inner table 6 and the outer table 5.

In another example, in the ICI 100 of FIG. 14E hereinafter, the induction coil 116, is disposed on or above the surface 5A of the outer table 5, and is not disposed between the inner table 5 and the outer table 6 of the calvarial bone.

Furthermore, the electronic circuitry module of some embodiments may be only in a passage or chamber made within the outer table 5 and the cancellous bone 7 of the calvarial bone. For example, a part or portion of the electronic circuitry module 24 of FIG. 10 is disposed within the opening formed in the outer table 5 and the remaining part or portion of the electronic circuitry module 24 is dispose within the hollow passage 7D that passes within the cancellous bone 7.

It is noted that the number of the electrodes 83 may be varied and any practical number of electrodes 83 having any size and/or shapes may be used to form the electrode array of the ICI 80. Typically, the number of the electrodes in the array may be in the range of 2-200 electrodes. However, the number of electrodes may be affected, inter alia, by the total area of the surface 81C, the minimal acceptable electrode size and/or surface area required for sensing and/or stimulation, the particular application type, and other practical engineering and manufacturing methods and considerations.

It is noted that in some embodiments of the systems and ICIs of the present application it may be preferred to dispose the power harvesting induction coil outside the ICIs and not within the calvarial bone. This may be because of several reasons. For example, if the required or desired size or diameter of the induction coil exceeds the dimensions of the space or hollow chamber available within the housing or the hollow chamber of the ICI, the placement of the induction coil of the system above the surface 5A of the outer table 5 (such as, for example, between the scalp and the outer table 5) may enable using a larger diameter coil (or a larger sized coil, if the coil being used is not circular) which may improve the energy harvesting by the implanted coil. Additionally, such a coil may be substantially closer to an external induction coil that is placed above the implanted induction coil because only the thickness of the scalp intervenes between the external energy transmitting coil and the implanted energy harvesting coil which may increase the power harvesting efficiency. Specific examples of such induction coils are disclosed in detail, inter alia, in FIGS. 13 and 14E, hereinafter.

Reference is now made to FIG. 13, which is a schematic part cross-sectional view of a system including an intra-calvarial electrode array an electronic circuitry module and a power harvesting coil that is disposed on the outer table of a calvarial bone, in accordance with an embodiment of the systems of the present application.

The system 90 may include an electrode array 98, an electronic circuitry module 24 and a power harvesting induction coil 96.

The system 90 is implanted in the skull bone or a calvarial bone by forming a hollow passage 7E in the calvarial bone using any of the methods disclosed herein. The passage 7E fully penetrates the outer table 5 and the cancellous bone 7 and also comprises a recess 6D made in the inner table 6 to reduce the thickness of the inner table 6 within the recess 6D, as disclosed in detail hereinabove.

The electrode array 98 may include a substrate 91 and multiple electrodes 93 attached to the substrate 91 or formed therein. The multiple electrodes may be attached within multiple recesses 98L formed in the substrate 91, as illustrated in FIG. 13. However, in some embodiments, the electrodes 93 may protrude beyond the surface 91A of the substrate 91 as disclosed in detail hereinabove. Alternatively, in some embodiments, the electrodes 93 may be plated on or deposited on or printed on or deposited on the surface 91A by using any suitable type of printing or plating or embossing, or coating or CVD or ion beam deposition or lithographic methods known in the art. In some embodiments, the substrate may be a rigid substrate, such as, for example a rigid biocompatible polymer based substrate (such as, for example, any suitable rigid polymer coated with a layer of Parylene®). In some other embodiments, the substrate 91 may be a flexible substrate made from any suitable flexible biocompatible elastomer, as described hereinabove. In some other embodiments, the electrode array may be any commercially available Ecog electrode array known in the art. An electrically insulated biocompatible band including multiple conductive elements 99 may be included in the electrode array 98 and may be used to electrically connect the electrodes 93 to the electronic circuitry module 24. Two electrically insulated electrically conducting wires 97A and 97B may be used for connecting the power harvesting coil 96 to the electronic circuitry module 24 in order to provide electrical power to the electronic circuitry module 24. In some embodiments, the electronic circuitry module 24 may be enclosed within a hollow housing (not shown) that may be made from any suitable material and may also be electrically shielded by a thin layer of gold that may be plated or coated or attached to such housing. Additionally or alternatively, the housing may be made from a suitable biocompatible material or may be coated with a layer of biocompatible material (such as, for example Parylene®).

In some embodiments, the induction coil 96 may have a curved surface 96A and a flat surface 96B. After the hollow passage 7E is made in the calvarial bone as disclosed hereinabove, the electrode array 98 may be inserted through the hollow passage 7E and placed into the recess 6D with the electrodes 93 facing the bottom surface 6F of the recess 6D. The electronic circuitry module 24 may then be inserted into the hollow passage 7E and the induction coil 96 may then be used to seal the opening of the hollow passage 7E by applying a sealant 55 to the flat surface 96B and to the surface 5A of the outer table 5 turning the hollow passage 7E into a sealed chamber.

Reference is now made to FIGS. 14A-14E which are schematic part cross-sectional views illustrating various steps of a method for forming extended laterally oriented hollow passages and/or hollow chambers within the cancellous bone layer of a calvarial bone, in accordance with some embodiments of the methods of implantation of the ICIs and systems of the present application.

Turning to FIG. 14A, a surgical drill 101 may be used for drilling through the outer table 5 and may then be advanced into the cancellous bone 7. The arrow 102 schematically indicates the direction of rotation of the drill 101.

Turning to FIG. 14B, the drill 101 may then be withdrawn from the calvarial bone after having formed an open hollow passage 103 within the calvarial bone.

Turning now to FIG. 14C, a laser boring tool 105 (only a part thereof is illustrated in FIG. 14C) may be inserted into the open passage 103 and advanced there along to the position illustrated in FIG. 14C. The laser boring tool 105 has a hollow shaft 105A through which a laser beam 111 may be directed. The laser beam 111 is reflected by a mirror 113 that may be attached to the shaft 105A at an angle of 45° to the direction of the laser beam 111. In accordance with some embodiments of the tool 105, the mirror 113 may be a fixed mirror. However, in some embodiments, the mirror 113 may be a rotatable mirror that may be rotated at various angles with respect to the longitudinal axis 105D of the hollow shaft 105A. The shaft 105A may be moved up and down the passage 103 in the directions schematically indicated by the double headed arrow 107 and may also be rotated within the passage 103 as schematically indicated by the double arrow 106. By suitably operating the tool 105, the laser beam 111 may be used to gradually ablate any material in its path (such as for example, parts of the cancellous bone 7, bone marrow and small branches of the diploic veins included within the cancellous bone 7) to gradually tunnel through the cancellous bone 7 to form either a hollow passage 120 extending roughly orthogonally to the passage 103 (as illustrated in FIG. 14C) or in non-orthogonal directions with respect to the passage 103. If the mirror 113 is rotatable, the rotating of the mirror 113 may add additional flexibility to the bone boring or bone sculpting laser beam 111 as compared to just using up and down movements of the shaft 105A and rotating the shaft 105A within the passage 103.

Turning now to FIG. 14D, after the bone sculpting performed by manipulating the laser beam 111, the tool 105 may be withdrawn out of the calvarial bone after having enlarged and/or extended the passage 103 to form a hollow chamber 109 within the cancellous bone 7. The chamber 109 may be of any desired shape and may also have one or several tunnels or contiguous open passages (not shown) extending in various directions from the central hollow chamber 109.

Turning now to FIG. 14E, after the bone sculpting process has been completed, and the tool 105 is withdrawn, an implantable system 100 may be implanted within the chamber 109. The system 100 may include a thin flexible and rollable electrode array 118, suitably electrically coupled to an electronic circuitry module 110 by a flat band 115A of insulated electrically conducting wires. The electrode array 118 includes multiple electrodes 120 attached to or formed in or formed upon a highly flexible elastic thin substrate 118A that may be made from or may include any of the biocompatible elastic flexible electrically insulating materials or elastomers disclosed in detail hereinabove.

It is noted that the electronic circuitry module 110 is shown only schematically in FIG. 14E for the sake of clarity of illustration. The module 110 may however be constructed as disclosed in detail hereinabove and hereinafter. The system 100 may also include a power harvesting induction coil 116 that may be similar to any of the induction coils disclosed herein. Preferably, the coil 116 may be a thin flexible single layer or multilayer induction coil, and may be suitably electrically connected to the electronic circuitry module 110 by a pair of electrically insulated electrically conducting wires 112.

During implantation of the system 100, the flexible electrode array 118 is rolled into a narrow spirally wound rounded elongated shape that fits within the narrowest diameter 103B remaining at the open end of the chamber 109. After insertion of the rolled electrode array through the opening 103B into the chamber 109, the rolled array is allowed to open and is spread over the lower surface 109A at the end of the chamber 109 such that the electrodes 120 are in contact with or in close proximity of the surface 109A (which may be formed on the inner surface 6B of the inner table 6 as illustrated in FIG. 14E). The opening 103B is then hermetically sealed by the electronic circuitry module 110 as disclosed hereinabove by using a suitable sealant (not shown in FIG. 14E for the sake of clarity of illustration. After the sealant hardens or sets properly, the induction coil 116 mat be carefully inserted under one of the scalp flaps 109A of the scalp 109 and positioned between the surface 5A of the outer table 5 and the scalp flap 109A. The flaps of the scalp 109 may be then allowed to come in contact with each other as illustrated in FIG. 14E and to cover the implanted electronic circuitry module 110 and the implanted induction coil 116. After healing of the scalp incision, the system 100 may be operated as explained in detail hereinabove and hereinafter.

It is noted that while the entire electrode array 118 of the system 100 is disposed within the chamber 109 (and between the inner table 6 and the outer table 5), only part or portion of the electronic circuitry module 110 is disposed within the chamber 109 and the passage 103B, while another part or portion of the electronic circuitry module 110 is disposed above the outer surface 5A of the outer table 5 and underneath the scalp 109.

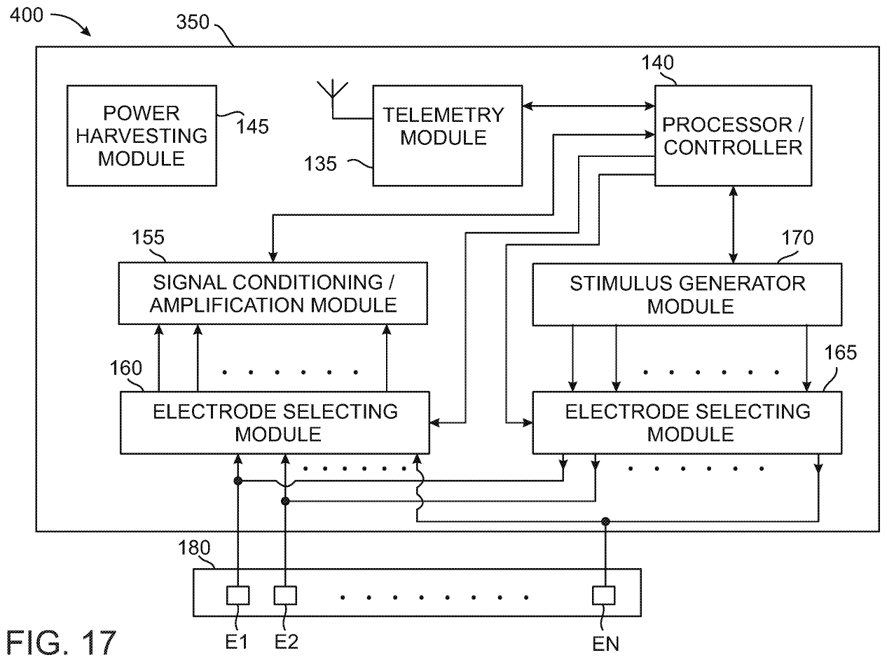

Reference in now made to FIGS. 15-17. FIG. 15 is a schematic block diagram illustrating some of the components of an intra-calvarial system for sensing and/or recording brain electrical signals, in accordance with some embodiments of the systems of the present application. FIG. 16 is a schematic block diagram illustrating some of the components of an intra-calvarial system for stimulating one or more regions of the brain, in accordance with some embodiments of the systems of the present application. FIG. 17 is a schematic block diagram illustrating some of the components of an intra-calvarial system for sensing/recording brain electrical signals and for stimulating one or more regions of the brain, in accordance with some embodiments of the systems of the present application.

Turning to FIG. 15, the system 200 may include an electronic circuitry module 150 and an ICI 180. The electronic circuitry module 150 may include an electrode selecting module 160, a signal conditioning/amplification module 155, a telemetry module 135, a power harvesting module 145 and a processor controller 140. The ICI 180 may be a multi-ICI that may include N electrodes E1, E2, E3, . . . , EN, wherein N is an integer number that may typically (but not obligatorily) be in the range of N=2-200, but in some embodiments in which high resolution electrode arrays are being used N may be significantly larger and may exceed several thousands of electrodes or even higher numbers. The above mentioned values of N should therefore be considered as typical and non-limiting and N may have much larger values depending, inter alia on the required resolution, the technology of producing the electrode array 180, the surface area of the electrode array 180, the ability to sense and transmit (and multiplex) N channels, the computational power of the processor/controller 140 and other engineering and manufacturing considerations.

Each of the N electrodes E1, E2, E3, . . . , EN, may be electrically operatively connected to the electrode selecting module 160, that may select from which electrode(s) or electrode combination electrical brain signals are going to be sensed. The electrode selecting module 160 is suitably connected to the processor/controller 140 that controls the operation thereof. The electrode selecting module 160 may be operatively coupled to the signal conditioning/amplification module 155 and may output the signals from the selected electrodes to the signal conditioning/amplification module 155 that may condition and amplify the signals received from the electrodes selected by the electrode selecting module 160.

The signal conditioning/amplification module 155 may be suitably connected to the processor/controller 140 which may control the operation thereof to determine and or change and/or select the type of desired signal conditioning depending on the specific application. In some embodiments in which the system 200 is designated to perform only a defined type of signal conditioning and amplification, the signal conditioning/amplification module 155 may be “hard wired” to perform a fixed and unchanging type of conditioning/amplification but some parameters (such as, for example the amplification gain and the filtering parameters) may be dynamically controlled by the processor/controller 140 based on the results of processing of the signals which may monitor the SNR and noise characteristics to control such parameters.

In some embodiments, the communication between the processor/controller 140 and other modules (such as, for example, the electrode selecting module 160 and the signal conditioning/amplification module 155 may be bidirectional communication lines, to enable these modules to send device status signals, error signals and possibly clock synchronization signals to the processor/controller 140.

The conditioning performed by the signal conditioning/amplification module 155 may include signal filtering such as, for example high pass filtering, notch filtering, low pass filtering or any other signal conditioning types required by specific applications. The conditioned/amplified signals may then be (optionally) stored in the memory of the processor/controller 140 and/or transferred to the telemetry module 135 for being transmitted to an external telemetry unit (not shown in FIG. 15) for further processing.

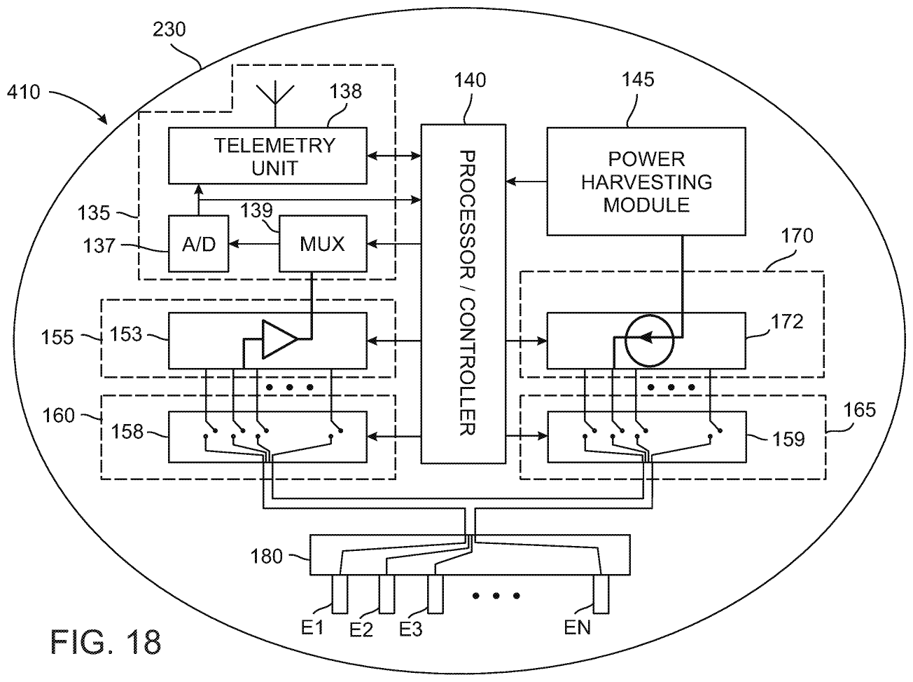

The telemetry module 135 may be configured to transmit either the (optionally processed) analog signals to an external telemetry unit or to digitized the signals and transmit digitized data to an external telemetry unit. The transmitter signals or data may have to be multiplexed for transmitting as disclosed hereinafter with respect to FIG. 18 hereinafter. The power harvesting module 145 may be any suitable power harvesting unit for providing harvested energy to any components or modules that need to be energized. The power harvesting unit may be implemented, for example, as an electromagnetic induction based power harvesting unit attachable to an implanted induction coil (such as any of the induction coils disclosed hereinabove) but may also be an ultrasonic energy harvesting unit including an implanted piezoelectric transducer that generates electricity when receiving energy from an external ultrasonic beam directed towards the implanted piezoelectric transducer. The power harvesting module may also be an electromagnetic radiation harvesting unit that includes an implanted antenna configured to receive electromagnetic waves transmitted from an external power transmitter. All suitable power harvesting methods known in the art that may provide sufficient energy for operating the systems disclosed herein may be implemented in the systems and ICIs of the present application.

It is noted that in all the drawing figures of the present application, the connections of the power harvesting unit(s) to all the modules and/or components in need of power are not shown in detail for the sake of clarity of illustration.

Thus, the system 200 may be used for sensing/recording of brain signals using any of the ICIs disclosed hereinabove, and for wirelessly sending the sensed recorded signals or data to an external device for further processing.

Turning now to FIG. 16, the system 300 may include an electronic circuitry module 250 and an ICI 180. The electronic circuitry module 250 may include an electrode selecting module 165, a stimulus generator module 170, a telemetry module 135, a power harvesting module 145 and a processor controller 140. The ICI 180 has been described in detail hereinabove with respect to the system 200.

Each of the N electrodes E1, E2, E3, . . . , EN, may be electrically operatively connected to the electrode selecting module 165, that may select to which electrode(s) or electrode combinations stimulating signals are going to be delivered by the stimulus generator module 170 that is operatively connected to the electrode selecting module 165. The electrode selecting module 160 is suitably connected to the processor/controller 140 that may control the operation thereof. The stimulus generator module 170 may also be suitably operatively connected to the processor/controller 140, for controlling the operation of the stimulus generator module 170. The processor/controller 140 may control the stimuli delivered to the electrodes of the electrode array 180 autonomously by executing an application program operating on the processor/controller 140. Alternatively, and/or additionally the processor/controller 140 may receive external instructions and/or control signals from an external control unit or module (not shown in FIG. 15) that may be communicating with the telemetry module 135 that is suitably operatively connected to the processor/controller 140. Such telemetrically received control signals may control or modulate or reprogram the operation of the processor/controller 140 and may be used to change or modulate or stop or begin the stimulation regime, through The telemetry module 135 may also be used for transmitting status signals and/or error signals from the processor/controller 140 to the external telemetry module or unit

The power harvesting module 145 may be any suitable power harvesting unit as disclosed in detail hereinabove for providing harvested energy to any components or modules of the system 300 that need to be energized.

Thus, the system 300 may be used for autonomously, or in interaction with an external control unit (not shown) deliver stimuli to any of the ICIs disclosed hereinabove, and for wirelessly interacting with an external device (such as any type of telemetrically capable controller device) for controlling or modulating the parameters of any stimuli delivered to selected electrodes or selected electrode combinations. Among the parameters controlled by the stimulating system 300 may be, the type of stimulating signals, the electrode(s) being stimulated, the stimulus amplitude, the stimulus shape and type such as for example the signal pulse parameters (monopolar pulses, bipolar pulses, monophasic pulses, biphasic pulses) the timing or frequency of stimulation, or any other type of stimulation parameter.

Turning now to FIG. 17, the system 400 may be a system capable of both sensing/recording brain electrical signals and also of stimulating the brain as disclosed hereinabove. The system 400 may include the electrode array 180 as disclosed hereinabove and an electronic circuitry module 350. The electronic circuitry module 350 may include the power harvesting unit 145, the telemetry module 135, the processor/controller 140, the signal conditioning/amplification module 155, the stimulus generator module 170, and the electrode selecting modules 160 and 165.

The processor/controller 140 may be operatively and bidirectionally connected to each of the telemetry module 135, the signal conditioning/amplification module 155, the stimulus generator module 17, and the electrode selecting modules 160 and 165 for controlling their operation as disclosed hereinabove.

Each of the N electrodes E1, E2, . . . EN, may be electrically coupled to the signal conditioning/amplification module 155 and the stimulus generator module 170 through the electrode selecting modules 160 and 165, respectively as illustrated in FIG. 17. Therefore, each of the selected electrodes or electrode combinations may be used for sensing/recording brain electrical signals and also for stimulating one or more brain regions.

In some embodiments, the system 400 may be used as a closed loop BCI system. In such a system, the signals sensed in the brain by the selected electrode(s) may be processed by the processor/controller 140 in to detect an indication of a physiological and/or neurological and/or neuropsychiatric of the brain and to automatically and/or autonomously deliver to the brain a selected stimulation regime responsive to the detection of such an indication.

In other embodiments, the system may sense and condition/amplify signals received from one or more brain regions as disclosed in detail with respect to the system 200. The system may then use the telemetry module 135 to telemetrically transmit the signals to an external control unit (not shown in FIG. 17) for processing by a processor/controller (not shown) included by such an external control unit that may have a higher computational power as compared to the processor/controller 140. The external control unit may perform the processing of the telemetrically received signals or data and may detect the indication of the physiological and/or neurological and/or neuropsychiatric state of the brain and responsive to such detection may telemetrically send appropriate, control signals to the processor/controller 140. Upon receiving the appropriate control signals the processor/controller 140 may initiate an appropriate stimulation regime for stimulating one or more brain regions to modulate the state of the brain in such a way that may result in changing or therapeutically treating the detected physiological and/or neurological and/or neuropsychiatric state of the brain.

For example, in a specific application to therapeutically treat mood disorders such as, for example, depression, the ICI 180 may be implanted for sensing electrical activity in the dorsolateral prefrontal cortex (DLPFC) to detect an indication that the patient is in a depressed state (such as for example modulation of High Gamma Band Power/Amplitude Changes may be monitored by sensing electrical brain signals in the DLPFC). When the power in the higher frequencies of the gamma band is below an empirically determined threshold value this may be an indication of a depressed state of the patient and the system may initiate stimulation of some target brain regions, such as, for example, the subgenual cingulate region (Brodmann area 25), the ventral capsule/ventral striatum (VC/VS), the Nucleus Accumbens, the Lateral habenula, the Ventral caudate nucleus (VCN), the Inferior thalamic peduncle, and/or any combination of the above brain regions.

Stimulation of such deep brain targets may be performed by the intra-calvarial implants and/or ICIs of the systems disclosed herein by using a Frequency Interference (IF) stimulation method similar to the TFIS method described by Grossman et al. with all the advantages conferred by the use of intra-calvarial electrodes as described in detail in the present application. Upon detection of such an indication (decreasing of the high frequency gamma band power below a threshold), the system 400 may also directly stimulate any some cortical regions (such as, for example, the DLPFC) hereinabove to treat the depression.

In another exemplary application, the system 400 may be used for enhancing cognition by sensing simultaneously in the DLPFC and the temporo-parietal cortex (TPC) and processing the sensed signals to detect an indication of enhanced phase locking between signals sensed in the DLPFC and the TPC in the beta frequency band. Upon detection of the indication, the system 400 may stimulate directly the DLPFC region which may result in enhanced cognitive performance of the user/patient.

In such a way the system 400 by using such closed loop BCI methods, may be used to treat many types of disorders, such as, for example, neurodegenerative disorders, neuropsychiatric disorder and/or psychiatric disorders, including, for example, epilepsy, traumatic brain injury (TBI), depression, obsessive-compulsive disorder (OCD), ADHD, attention deficit disorder (ADD), eating disorders including bulimia and anorexia, obesity, and other types of disorders.

Reference is now made to FIG. 18 which is a schematic block diagram illustrating an exemplary system for sensing/recording brain electrical signals and for stimulating one or more regions of the brain, in accordance with some embodiments of the systems of the present application.

The system 410 may include an ICI including an electrode array 180. Any of the electrode arrays included in any of the ICIs disclosed in the present application may be used. The electrodes E1, E2, E3, . . . , EN of the electrode array 180 may be suitably electrically coupled to the electrode selecting modules 160 and 165 as illustrated in FIG. 18. In the specific embodiment of FIG. 18, the electrode selecting unit 160 is implemented as a first solid state switching device 158, and the electrode selecting unit 165 is implemented as a second solid state switching device 159. The signal conditioning/amplification module 155 is implemented as a solid state multi-channel 153. The stimulus generator module 170 is implemented as a solid state multi-channel stimulator 172.

The telemetry module 135 may be implemented by a telemetry unit 138 a multiplexer (MUX) 139 and an analog to digital converter (A/D) 137.

The multiple amplified signals outputted from the multi-channel amplifier 153 may be multiplexed by the MUX 139, digitized by the A/D 137 and fed to the telemetry unit 138 to be transmitted to an external telemetry receiver as disclosed hereinabove. The multiplexing may be implemented as any type of multiplexing method known in the art, including but not limited to time division multiplexing, and frequency division multiplexing. The transmitted signals may be suitably decoded by the external telemetry unit or module and the data may be processed by a processor/controller (not shown in FIG. 18) coupled to the external telemetry unit or module. The external processor/controller may detect an indication as disclosed in detail hereinabove and may telemetrically control the processor/controller 140 to initiate stimulation by delivering suitable stimulating signals to selected electrodes of the electrode array 180.

It is noted that the type of stimulation generated by the multi-channel stimulator 172 may be either direct stimulation of selected cortical regions, and/or a frequency interference (FI) type of stimulation at two different high frequencies as disclosed in detail by Grossman et al. This type of intra-calvarial FI stimulation may enable stimulation of selected deep brain structures but may benefit from the intra-cranial placement of the electrodes which may advantageously result in improved stability and repeatability of the stimulation due to the closer distance of the electrodes of the electrode array 180 to the surface of the brain and the removal of the above mentioned problems of using external EEG type electrodes as disclosed by Grossman et al. Moreover, the use of the disclosed intra-calvarial electrodes disclosed in the present application may require reduced stimulation amplitudes (or intensities) that may advantageously save energy stored of in the implanted power harvesting modules of the ICIs of the present application. The reduced stimulation amplitudes may result, inter alia, due to the smaller electrical impedance of the inner table 6 in stimulation by intra-calvarial electrodes as compared to the much higher electrical impedance of the entire thickness of the scalp and the calvarial bone interposed between the brain and the stimulating electrodes disclosed by Grossman et al.

Furthermore, due to the ability to select many type of differently spaced and differently positioned electrode pairs selectable for stimulation by the solid state switches 158 and 159, the flexibility repeatability and finesse of the focal brain region stimulation using intra-calvarial electrode arrays may be significantly enhanced.

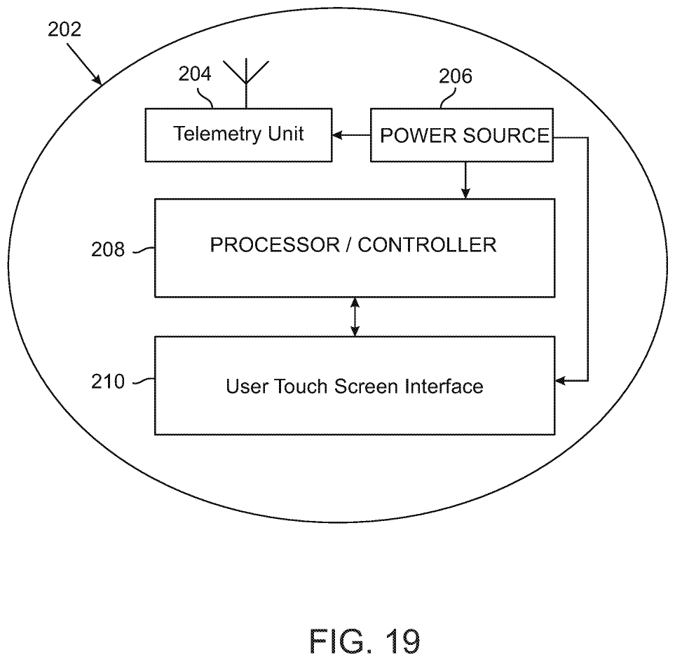

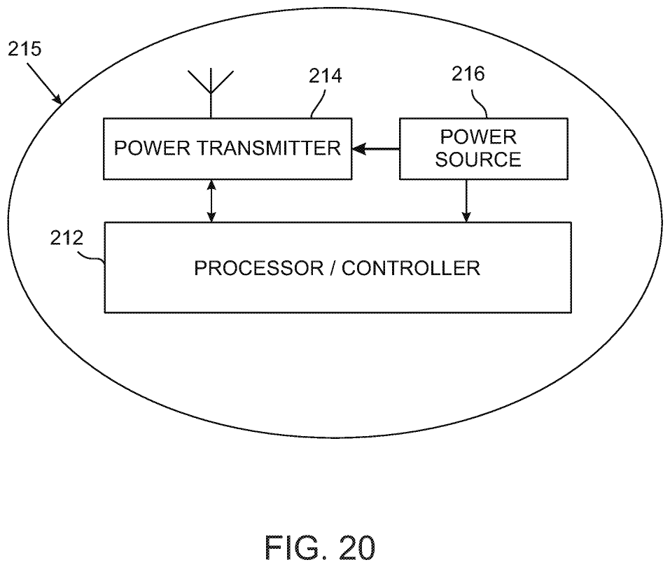

Reference is now made to FIGS. 19-20. FIG. 19 is a schematic functional block diagram illustrating some components devices of a hand held or wearable or portable device usable as part of the systems of the present application. FIG. 20 is a schematic block diagram illustrating some components of an energizing device usable for providing power to some of the systems and/or ICIs of the present applications.

Turning to FIG. 19, in accordance with some embodiments an external controlling/processing device 202 may be included in the systems of the present application. The device 202 may be a mobile and/or portable and/or hand held device, and/or wearable device such as, for example, a mobile (cellular) telephone or smartphone, a pablet, a tablet, a laptop computer a notebook or a wearable virtual reality (VR) headset, or a wearable augmented reality (AR) headset. The device 202 may include a processor/controller 208 (such as, for example the processor included in a cellular telephone). The device 202 may also include a power source 206 (such as, for example the rechargeable lithium battery of a cellular telephone). The device 202 may also include a telemetry unit 204 (such as, for example, the cellular transmitter of the smartphone or cellular telephone) that is suitably connected to and controlled by the processor/controller 208. The device 208 may also include a user touch screen interface 210 (such as, for example, the touch sensitive display screen of a cellular telephone or smartphone that enables the user to interact with any application operating on the processor(s) included in such mobile cellular telephones).

It is noted that, the external controlling processing device 202, is not limited to mobile phones or smartphone but may also be implemented as any device having wireless communicating capabilities processing power and a user interface (physical and/or virtual), including for example, a laptop or other computer, a notebook, a pablet, a virtual reality(VR) headset, such as, for example, virtual reality goggles, VR eyeglasses, augmented reality (AR) headsets, or any other device enabling the user to interact with a virtual or physical user interface of any type.

In operation, a software program or mobile application may be installed on the smartphone that may wirelessly receive data from any of the systems (such as, for example any of the systems 20, 300, 400 and 410 disclosed hereinabove) and may process the received data to detect an indication of a change in the state of the user in which the intra-calvarial electrodes or ICIs are implanted, as disclosed hereinabove. The processor(s) of the telephone may telemetrically send control signals to the telemetry module 135 of any of the systems being used that when received by the processor controller 140 may initiate a stimulation regime of one or more brain regions of the user/patient by controlling the stimulus generator module 170 and/or the electrode selecting module 165 of the systems 300, 400 and 410. In such a way, the telephone may be included as part of the system 400 and/or 410 which together may operate as a BCI system.

Furthermore, the inclusion of such a device 204 or the telephone, may enable further interactions of the user/patient with the systems by using the user touch screen interface 210 for displaying data or processed date on the screen representing the status of the system, the parameters of stimulation and other information about the status and operational state and/or operational history of the system. In some embodiments the user or the patient may be able to feed data or control instructions to the touch screen interface 210, for changing and/or modulating, and/or stopping, and or initiating the operation of the system.

It is noted that the user interface of the portable and or mobile and/or wearable devices disclosed hereinabove may be any type of physical interface (such as, for example, a keyboard, a touch-screen) but may also be any other type of interface, such as a graphic user interface (GUI), a virtual GUI, or any other type of suitable user interface.

Turning now to FIG. 20, the power transmitting device 215 may be used with or may be included in any of the systems disclosed in the present application for providing power to the power harvesting modules 135 of any of the systems. The device 215 may be a hand held or a portable device. The device 215 may include a power source, such as, for example, a battery or electrochemical cell or rechargeable battery or any other suitable power source. The power source 216 may suitably connected to a processor/controller 212 and to a power transmitter 214. For example, if the power harvesting module 145 used by the system is connected to an induction coil (such as any of the different induction coils disclosed in the present application), the power transmitter 214 may be configured to include an external induction coil (schematically represented by the antenna symbol illustrated on the power transmitter 214) that may be placed near or in close proximity to the implanted induction coil under the scalp of the user/patient to effectively transmit electrical power to the implanted induction coil.

This transmitted power maybe rectified by suitable electronic circuitry (not shown) in the power harvesting system and may be stored in any suitable type of charge storage device (such as, for example, a rechargeable electrochemical cell or a super-capacitor) included in the power harvesting module of the system (such as, for example the power harvesting module 145) and may be used for powering the components of the system (such as, for example, the systems 200, 300, 400 and 410 disclosed herein).

It is noted that the device 202 and the power transmitting device 215, need not obligatorily be separate devices. For example, the components and functionalities of the device 202 and of the power transmitting device 215 may be combined in a single device (not shown) such that the combined device may be used for controlling the ICI(s), storing and/or processing data received from the ICI(s), transmitting or offloading stored data and/or stored processed data into other devices (such as, for example, computers, laptops or servers, in communication with such a combined device) and also for transmitting power to the ICI(s). Preferably (but not obligatorily), such a combined device may be a hand held compact device so that it may be easily handled by the patient to provide power to the ICI(s)

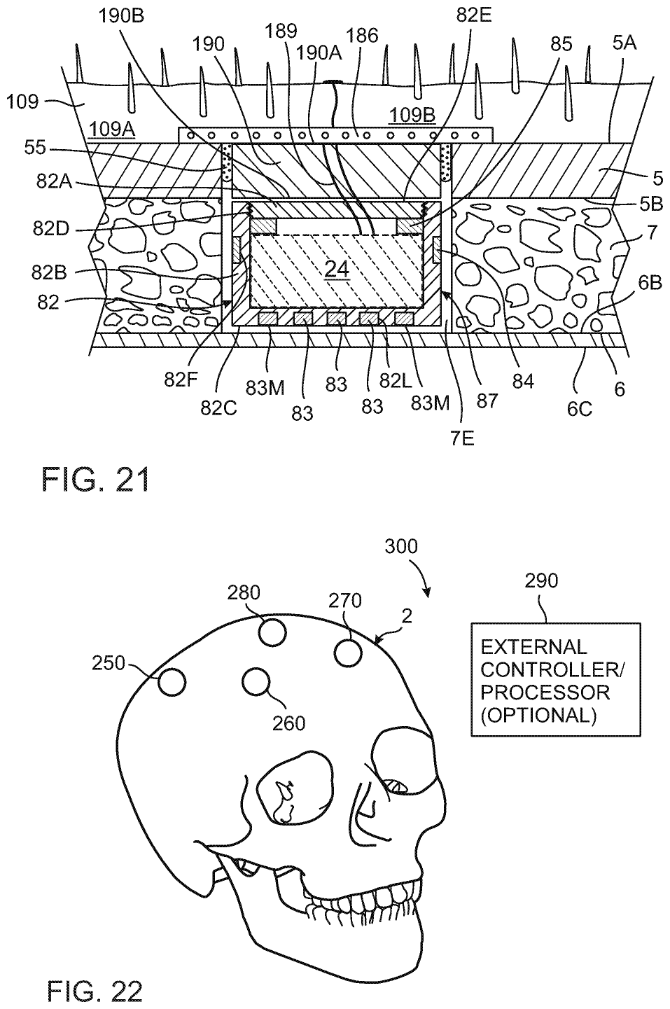

Reference is now made to FIG. 21 which is a schematic cross-sectional diagram illustrating an intra-calvarial implant that is fully disposed within the cancellous bone layer of the calvarial bone of the skull, in accordance with some embodiments of the implants of the present application. The ICI 87 has a housing comprises a hollow cylindrical housing 82 having a cylindrical base member 82B and a flat lid 82A. In some embodiments, the base member 82B and the lid 81A may be made from a strong electrically insulating material such as for example, Kevlar® or any other suitable biocompatible material and may be coated by a thin layer of biocompatible material such as, for example, Parylene®. The lid 82A has a threaded portion 82D that may be tightly screwed into a matched female thread formed in the upper open end of the base member 82B to hermetically seal the housing 82.

The ICI 87 may also include the electronic circuitry module 24, an annular spacer 85. The portion of the base member 82B facing the outer surface 6C of the inner table 6 includes multiple electrodes 83 that may be attached within suitable recesses 82L formed in the surface 82C of the base member 82B. The electrodes 83 may be formed from any suitable electrically conducting material as disclosed in detail hereinabove. The electrodes 83 may be attached within the recesses 82L such that the surfaces 83M of the electrodes 83 are flush with the surface 82C of the base member 82B. Alternatively, in some embodiments, the surfaces 83M of the electrodes 83 may protrude from the surface 82C. Each of the electrodes 83 may be suitably electrically connected to the electronic circuitry module 24 by an insulated electrically conducting wire (the wires are not shown in FIG. 21 for the sake of clarity of illustration).

In accordance with some embodiments of the ICI 87, the housing 82 may also include a reference electrode 84 that may be implemented as a suitably electrically conducting annular member attached within a suitable recess 82F made in the upper part of the cylindrical base member 82B. The reference electrode 84 may be suitably electrically connected to the electronic circuitry module 24 by suitable electrically conducting wires (not shown in FIG. 21 for the sake of clarity of illustration). However, this arrangement is not obligatory and the reference electrode may also be implemented in a different location and/or a different structure. For example, in some embodiments of the ICIs, the reference electrode may be implemented as one or more of the electrodes 83, in another example, a reference electrode may be attached to the lower surface 190B of the shim 190 and may be electrically connected to the electronic circuitry module 24 by an insulated electrically conducting wire (not shown in FIG. 21 for the sake of clarity of illustration).

It is also noted that the reference electrode 84 may be used for sensing/recording by performing differential recording of the voltage between the reference electrode 84 and any electrode selected from the multiple electrodes 83. However, the electrode 84 may also be used for stimulation. During stimulation, one or more current pulses may be passed between one or more electrodes selected from the multiple electrodes 83 and the electrode 84. In such a stimulation, the electrode 84 may be referred to as a return electrode 84 or ground electrode 84.

It is noted that although the electrode 84 may be interchangeably used as a reference electrode in sensing/recording and as a return electrode (or ground electrode) for stimulation, this is by no means obligatory. For example, any selected pair of electrodes may be used for sensing/recording signals and/or for stimulation of brain tissues. For example, sensing and/or stimulating may be performed by performing differential recording between any two selected electrodes 83 or by passing current between the two selected electrodes 83, respectively.

Moreover, returning briefly to FIGS. 15-18, by suitably using electrode selecting circuitry (such as, for example the electrode selecting modules 160 and 165 of FIGS. 15-18), it may be possible to selectively simultaneously combine one or more of the electrodes E1, E2, E3 . . . EN to form a first subset of electrodes and to combine one or more electrodes from the remaining electrodes to form a second subset of electrodes. The first and second electrode subsets may then be used for stimulating by passing current between the first subset of electrodes and the second subset of electrodes. The first and second electrode subsets may also be used for sensing/recording the voltage difference sensed between the electrodes of the first subset and the electrodes of the second subset. Therefore, one of the advantages of using electrode arrays in combination with electrode selecting modules is the high degree flexibility in performing recording and stimulation between selected combined electrode subsets and the ability to vary the location of the selected subsets on the electrode array and also to control the combined surface area of the electrode subsets.

The arrangement of the electrodes 83 on the surface 82C of the housing 82 forms an electrode array that is integrated into the base member 82B. The advantage of such a mono-block configuration is that it allows shortening the distance between the electrodes 83 and the electronic circuitry module 24 with concomitant shortening of the length of the wires which may improve the SNR as disclosed hereinabove.

When the ICI 87 is implanted in a skull, the implantation method may include making an incision in the scalp overlying the outer table 5 of the calvarial bone (or any other skull bone) to expose the surface 5A of the inner table 5 of the bone, forming a cylindrical hollow passage 7E passing through the outer table 5 and all of the cancellous bone 7. In accordance with some embodiments, the passage 7E may include a recess 6D (as illustrated in FIG. 12) that is formed within the inner table 6 in order to reduce the thickness of the dense bone of the inner table 6 within the recess 6D.

After the passage 7E is formed, ICI 87 may be inserted into the passage 7E until the electrodes 83 are in contact with or in close proximity to the surface 6B. Optionally, the entire ICI 87 may be rotated while testing the sensing/recording and/or stimulating of the ICI 80 to optimize the positioning and orientation of the electrodes 83 for adjusting the effects of stimulation and/or sensing/recording (which may be necessary, in embodiments in which the electrodes 83 are arranged in an elongated type of array configuration).

A feature of the ICI 87 is that the dimensions of the ICI 87 are such that the entire housing 82 of the ICI 87 is disposed within the layer of cancellous bone 7 of the calvarial bone to reduce electrical noise and improve the SNR. Therefore, after the ICI 87 is inserted into the hollow opening 7E, the upper surface 82E of the lid 82A is disposed below the plane defined by the inner surface 5B of the outer table 5. A shim 190 may be attached (by gluing or by any suitable attachment mechanism, such as, for example, by including a male bayonet type attachment member in the part of the shim 190 facing the lid 82 and forming a suitable matching female bayonet type recess in the upper surface 82E of the lid 82A, such glue or attachment mechanism are not shown in FIG. 21 for the sake of clarity of illustration) to the surface 82E of the ICI 87 such that the upper surface 190A of the shim 190 is flush with the surface 5A of the outer table 5. The shim 190 may be made from a biocompatible material such as, for example Parylene® or any other suitable biocompatible material. The shim 190 may be selected from a suitable set (or kit) of shims having various different thicknesses to enable the surgeon to make the surfaces 5A and 190A flush. The shim 190 may have suitable narrow passages (not shown for the sake of clarity of illustration) formed therein through which the pair of wires 189 may pass. The pair of electrically conducting insulated wires 189 may be suitably electrically coupled or connected to an induction coil 186 for providing electrical power to the electronic circuitry module 24 for the operation thereof.

After the attaching of the shim 190 to the ICI 87, a suitable biocompatible sealant 55 may then be used to seal the opening of the passage 7E, as illustrated in FIG. 21. After the sealant 55 sets and/or hardens, the pair of wires 189 may be electrically connected to an induction coil 186, the induction coil 186 is placed on the surface 5A of the outer table and possibly (but not obligatorily) over the shim 190, the skin flaps 109A and 109B of the incision in the scalp 109 may be closed to allow the scalp to cover the implantation region the scalp may be allowed to heal.

The induction coil 186 useable for power harvesting as disclosed in detail hereinabove may be a multilayer/multi-winding type of coil as illustrated in FIG. 11, but may also be, in some embodiments, a single layered induction coil (planar or curved, as disclosed in detail hereinabove).

Beyond a single implant configuration, the present application also envisions and contemplates the implantation and use of multiple implants that may also have unique capabilities. First, more than one ICI may be implanted at different locations in the skull. This may enable stimulation or inhibition of multiple brain regions to augment or further tailor a functional effect. Specific examples may include stimulating multiple nodes of a functional network (e.g. for the attentional network both parietal lobe and frontal lobe sites may be concurrently stimulated).

Alternatively, different brain networks may be co-stimulated for complimentary effects. As an example, both attentional and motor networks may be stimulated to enhance the performance in a professional athlete. An embodiment of the methods for stimulating multiple brain regions may combine both neuro-modulation regimes that stimulate one region and inhibit another region to create a tailored functional experience. As an example of such embodiments of the present application, the dorsolateral prefrontal cortex (DLPFC) may be stimulated while pre-frontal inhibitory regions may be inhibited to globally enhance attention and focus.

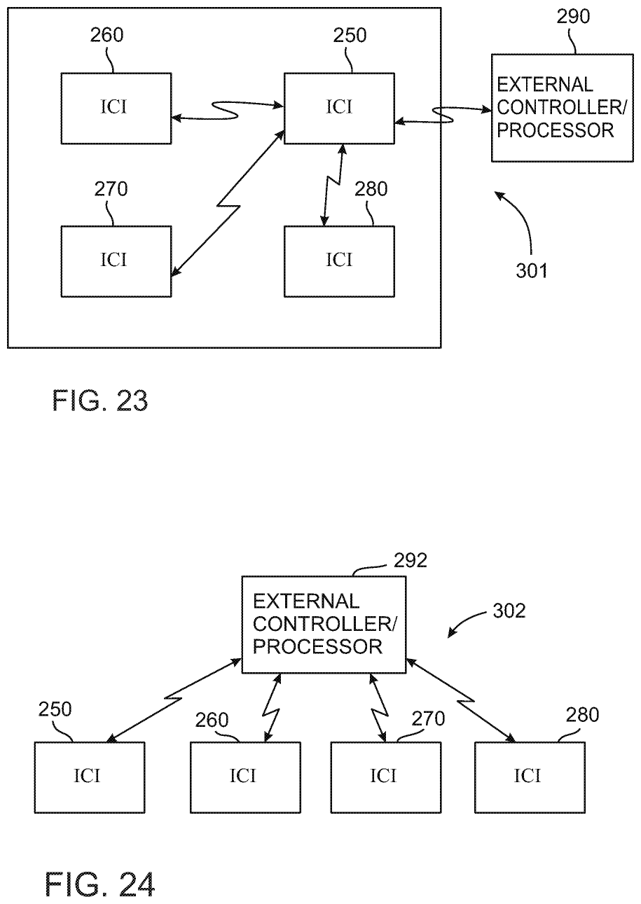

Reference is now made to FIGS. 22-24. FIG. 22 is a schematic isometric view illustrating a system including four ICIs implanted in the skull of a patient in accordance with some embodiments of the systems of the present application. FIG. 23 is a schematic functional block diagram illustrating the communication scheme of a first configuration of the system components of FIG. 22, in accordance with some embodiments of the systems of the present application. FIG. 24 is a schematic functional block diagram illustrating the communication scheme of a second configuration of the system components of FIG. 22, in accordance with some embodiments of the systems of the present application.

Turning to FIG. 22, the system 300 may include four ICIs 250, 260, 270 and 280, and an (optional) external controller/processor 290. The ICIs 250, 260, 270 and 280 may be implemented as any of the ICIs disclosed and illustrated in the present application. In some embodiments of the system, all the ICIs 250, 260, 270 and 280 may be of the same type. In accordance with some other embodiments of the system the ICIs 250, 260, 270 and 280 may be of different types (in a non-limiting example, the ICIs 250 and 280 may be of the type illustrated in FIG. 12 and the ICIs 260 and 270 may be of the type illustrated in FIG. 21). However, it is noted that any combination of any of the types of ICIs disclosed in the present application may be used in the system 300. The system 300 may also include an external controller/processor 290.

The external processor/controller 290 may be any external device positioned outside the body of the patient that has wireless communication capabilities and signal and/or data processing capabilities. For example the controller/processor 290 may be implemented as the device 202 of FIG. 19 or as the power transmitting device 215 of FIG. 20 or as the combine device including power transmitting components and communication and processing components, as disclosed in detail hereinabove.

As disclosed hereinabove with reference to FIGS. 15-20, the ICIs 250, 260, 270 and 280 may each include a processor/controller (such as, for example the processor/controller 140 of FIGS. 15-18) and other components necessary for recording signals from the brain region underlying the calvarial bone and for stimulating cortical regions and possibly deep brain structures as disclosed in detail hereinabove for the ICIs of FIGS. 5-13. Such components may include, for example sensing/recording electrodes, stimulating electrodes and/or stimulating/recording electrodes, signal conditioning and amplification modules, electrode selecting module(s), and stimulus generating module(s) as disclosed hereinabove and illustrated, inter alia, in FIGS. 15-18. The ICIs 250, 260, 270 and 280 may each include a telemetry module (such as, for example, the telemetry module 135 of FIGS. 15-17 or the telemetry module 138 of FIG. 18).

Turning to FIG. 23, the system 301 is an embodiment of the system 300 in which the controller/processor units of each of the ICIs 260, 270 and 280 are programmed to bidirectionally wirelessly communicate (through their respective telemetry modules) with the telemetry module of the ICI 250. The controller/processor of the ICI 250 is programmed to bidirectionally wirelessly communicate with the ICIs 260, 270 and 280 through the telemetry module thereof. The controller/processor of the ICI 250 is also programmed to bidirectionally wirelessly communicate with the external controller/processor 290 through the telemetry module thereof.

In operation of the system 301, when the system 301 is used to sense/record electrical activity in cortical regions and to stimulate cortical and/or deep brain regions using the ICIs 250, 260, 270 and 280, the recorded signals from the sensing electrodes of any of the ICIs 260, 270 and 280 may be wirelessly communicated to the controller/processor of the ICI 250 for storage and/or processing. After storage and/or processing of the sensed/recorded signals or data, the ICI 250 may wirelessly communicate the stored sensed signal/data and/or the processed data from all the ICIs that performed sensing (including the ICI 250, if the ICI 250 is also used for performing sensing) to the external controller/processor 290. If the data received by the external controller/processor 290 needs processing or further processing, the external controller/processor 290 may process or further process the data for generating any necessary stimulation control signals.

The generated control signals may be wirelessly transmitted by the telemetry unit included in the external controller/processor 290 to the wirelessly ICI 250. The ICI 250 may then wirelessly transmit stimulating control signals to the relevant ICIs in order to perform synchronized stimulation of the brain by the relevant ICIs. If the ICI 250 itself is also used for stimulation, the ICI 250 may be programmed to synchronize its internally generated stimulation control signals with the control signals transmitted wirelessly to other ICIs and to take into account any delays caused by the wireless transmission time and any processing time required by the ICIs to which such stimulation control signals are being transmitted. It is noted that in the system 301, the ICI 250 may be referred to as a “master ICI” and the ICIs 26, 270 and 280 may be referred to as “Slave ICIs”.

The advantages of using the system 301 as disclosed hereinabove, may include, inter alia, simplifying the communication scheme used by the external controller/processor 290 as the telemetry module of the external controller/processor 290 may need to communicate only with a single ICI, and saving power for the ICIs 260, 270 and 280 as they may use reduced power in communicating with the ICI 250 due to the shorter distances between the ICI 250 and the ICIs 260, 270 and 280. However, the ICI 250 may still require more power for communication with the external controller/processor 290 than with the ICIs 260,270 and 280 as the distance between the external controller/processor 290 and the ICI 250 may be longer as compared to the distances between the ICI 250 and the ICIs 26, 270 and 280).

Turning to FIG. 24, the system 302 the system 302 is an embodiment of the system 300 in which the controller/processor units of each of the ICIs 250, 260, 270 and 280 are programmed to bidirectionally wirelessly communicate (through their respective telemetry modules) with the telemetry module of the external controller/processor 290. The controller/processor of the external controller/processor 290 is programmed to bidirectionally wirelessly communicate with each of the ICIs 250, 260, 270 and 280 through the telemetry module thereof.

In operation of the system 302, when the system 302 is used to sense/record electrical activity in one or more cortical regions and to stimulate one or more cortical and/or deep brain regions using the ICIs 250, 260, 270 and 280, the recorded signals from the sensing electrodes of any of the ICIs 250, 260, 270 and 280 may be wirelessly communicated to the external controller/processor 290 for storage and/or processing. The external controller/processor 290 may wirelessly communicate the stored sensed signal/data and/or the processed data from all the ICIs that performed sensing to the external controller/processor 290. The external controller/processor 290 may process or further process (in case the sensed signals are partially processed by the respective processor/controllers of the ICIs) the data received from the ICIs for generating any necessary stimulation control signals. The generated control signals may be wirelessly transmitted by the telemetry unit included in the external controller/processor 290 to one or more of the ICIs 250,260, 270 and 280 in order to perform stimulation of the brain by the relevant ICIs.

The advantages of the system 302 may be, inter alia, reducing power requirements due to no or minimal processing of the sensed signals by the ICIs 250, 260, 270 and 280, and simplifying the programs operative on the ICIs (at least by simplifying the program operative on the ICI 250, by eliminating the need for receiving and/or storing data from the remaining ICIs 260, 270 and 280 and for transmitting of stimulation control signals from the ICI 250 to the remaining ICIs participating in stimulation.

It will be appreciated that in all of the multi-ICI systems 300, 301 and 302 many different configurations may be implemented. For example, in such systems, one or more of the ICIs 250, 260, 270 and 280 may be used for sensing, and one or more of the ICIs 250, 260, 270 and 280 may be used for stimulation. Generally, any combinations of sensing and stimulating may be used. For example, in some embodiments all of the ICIs 250, 260, 270 and 280 may be used for both sensing and stimulating. In other embodiments, one ICI may be used for sensing while the remaining ICIs may be used for stimulating. In other embodiments, two ICIs may be used for sensing and two or more ICIs may be used for stimulating. In short, any desired number of ICIs selected from the ICIs 250, 260, 270 and 280 may be used for sensing and/or for stimulation depending, inter alia, on the site of implantation, the type of stimulation being used, such as, for example, stimulation by passing current pulses between different electrodes of a single ICI, or stimulation using the frequency interference (FI) method as disclosed hereinabove using any electrode combinations of one or more ICIs.

It is also noted that while the systems 300-301 and 302 include four ICIs, this is by no means limiting and other embodiments of multi-ICI systems may be implemented with any desired number of ICIs (typically, in the range between 2-10 ICIs, but ICI numbers higher than 10 may also be implemented, depending, inter alia, on the specific application, the dimensions of the ICIs being used, the type of stimulation required and other considerations). In some embodiments of such multi-ICI system the external controller/processor (such as for example the external controller/processor 290) may directly wirelessly communicate with all the ICIs of the multi-ICI system for receiving data therefrom and for transmitting control signals thereto. In other embodiments of such multi-ICI systems, the external controller/processor (such as for example the external controller/processor 290) may wirelessly bidirectionally communicate with one (or possibly, more than one Master type ICIs while the remaining ICIs are of the slave type which may wirelessly bidirectionally communicate with such one or more master type ICIs.

Beyond using variable direct stimulation of cortical regions, the location and timing of stimulation could be configured to stimulate or inhibit deeper regions of the brain. By delivering to the brain multiple electric fields at frequencies too high to recruit neural firing, but which differ by frequency within the dynamic range of neural firing, one can electrically stimulate neurons throughout a region where interference between the multiple fields results in a prominent electric field envelope modulated at the difference frequency. The methods for performing such frequency interference (FI) stimulation are disclosed in detail in the article by Grossman et al. referenced hereinabove.

International publication No. WO 2018/109715 discloses the use of BCIs for sensing and stimulation of cortical regions and/or deeper brain structures using Ecog electrode arrays and other recording/stimulating surface electrodes for enhancing intelligence. The sensing, data processing and stimulation methods used in WO 2018/109715 may also be adapted for use with any of the ICIs and ICI systems disclosed in the present application, by using the ICIs disclosed herein instead of (or in addition to) the Ecog arrays and/or FI scalp electrodes disclosed in WO 2018/109715.

Intra-calvarial recording from and/or stimulating the cortical surface of the brain may enable neuro-modulation of electrophysiology that may have a wide range of clinical and non-clinical applications. In clinical applications of some embodiments, the cortical stimulation may be used to modify cortical excitability to treat numerous neuropsychiatric diseases such as, but not limited to, depression, ADHD, addiction, and obesity. From a purely recording standpoint, cortical signals may be used for brain computer interfaces that may be used to treat a wide array of motor disabilities. From a non-clinical perspective, modulating the brain physiology either through stimulation or recording methodologies can be used to enhance cognitive function. Depending on the modality, the location in the brain, and the interface regime, cognitive operations such as attention, memory, analytic abilities, and mood may all be enhanced beyond a given individual's normal baseline.

A barrier for more wide spread adoption of these type of approaches is the invasiveness of the implantation of the electrodes. Once the skull bone and dura mater are penetrated with either intra-parenchymal or electrocorticographic electrodes there is a risk of having an intracranial hemorrhage or infection that could cause major harm, morbidity, or even death. While these risks, generally speaking, are quite small, the mere fact that they exist substantially changes a patient's perception of considering adoption of such invasive procedures. This also changes the manner in which patients are treated by physicians after implantation. If an intracranial electrode implant is surgically placed (e.g. deep brain stimulator, cortical stimulator, deep brain stimulator etc.), at the very least the patients are kept overnight for observation in a hospital to ensure that, should an intracranial complication arise, it can be rapidly addressed. A major need is to have a recording and stimulation brain interface that has minimal risk of an intracranial complication yet is still able to record and stimulate the brain with a functional equivalence similar or very close to that of the intracranial approaches. The presently disclosed ICIs electrode systems, and methods of their construction and use may effectively and safely address many of the problems of the currently used intracranial methods.

It is noted that the processor/controllers disclosed herein may be or may include one or more computing devices selected from, one or more intracranial processor/controller, wearable processor/controller, remote processor/controller, a digital signal processor (DSP), a graphic processing unit (GPU), a quantum computing device, a central processing unit (CPU), or any combinations of the above. In some embodiments, the processor/controller may include and/or emulate a neural network. For example, the processor/controller(s) 140 or any other processors/controllers disclosed herein may include or may be connected to one or more neuromorphic ICs. Alternatively and/or additionally, the processor/controller 140 or any other processors/controllers disclosed in the present application may be programmed to emulate one or more neural networks by software operative on the processor/controller(s).

Furthermore, any of the processor/controllers disclosed in the present application may have access to the “cloud” via the internet (preferably, wirelessly, but also possibly in a wired way) or through any other type of network, such as, for example, a LAN, a WAN, a VPN or any other type of wired or wirelessly accessible network.

In some embodiments, the processor/controller(s) disclosed herein may include wireless communication circuits, such as Bluetooth, or WiFi communication units or circuits (not shown in detail any of the figures for the sake of clarity of illustration). Such wireless communication means may enable the processor/controller(s) to wirelessly communicate with external devices, such as for example, a remote computer, a server, a cellular telephone, a laptop computer, a VR headset, an AR headset or any other type of device having wireless communication capabilities (such as, for example, the device 202 of FIG. 19. Such embodiments may be useful in cases in which the processing power of the processor/controller(s) is limited. Such embodiments may allow the offloading of some or all of the computational burden to other processing devices, such as remote computer(s), servers, a cluster of computers, cellular smartphones, cellular telephones, a Pablet, a tablet or any other suitable computing devices, and may enable the use of cloud computing, or parallel computing for processing data.

It is noted that in some embodiments of the ICIs of the present application, the implanted ICIs and/or other components of the system disclosed herein may be made MRI-compatible to enable use of the implanted ICIs during performing of fMRI imaging procedures. This may be performed by selecting non-magnetic and/or non-magnetizable materials to be used in the construction of the components of such ICIs. Such material may include, as an example, titanium, organic polymers or polymer based materials such as, Kevlar®, Parylene® and other suitable materials. In such MRI-compatible embodiments, the power harvesting induction coils may be replaced by other types of power harvesting modules, such as, for example ultrasound energy harvesting modules using an implanted piezoelectric element coupled to the power harvesting units of the ICI. The use of fMRI compatible ICIs may be advantageous because it may allow the performing of complementary fMRI imaging to monitor the operation of the implantable system disclosed herein during implantation, testing and/or calibrating of the systems, as well as for tuning and/or adjusting the sensing and/or the stimulating regimes to improve or optimize system's performance. Additionally, the performing of fMRI imaging may assist the procedure of implantation, as disclosed in detail hereinabove.

In some embodiments of the systems and methods disclosed herein, the patient may undergo both anatomic imaging and functional imaging to define the optimal placement of the implanted ICI. For example, patients may be scanned using 3-T MRI scanners (such as but not limited to 3-T MRI scanners commercially available from Siemens, Erlangen, Germany). Anatomic imaging may include T1-weighted magnetization-prepared rapid acquisition gradient echo (MP-PAGE), T2-weighted fast spin echo, susceptibility-weighted imaging (SWI), diffusion-weighted imaging (DWI) and pre and post gadolinium T1-weighted fast spin echo in multiple projections. fMRI data may be acquired, for example, by using a T2*EPI sequence (3×3×3-mm³ voxels; 128 volumes/run; TE=27 ms; TR=2.8 s; field of view=256 mm; flip angle=90°), while the patients are instructed to remain still and fixate on a visual cross-hair without falling asleep (2 runs of 6 minute each for a total time of 12 minutes). Additionally, patients may also undergo CT scans with bone windows to determine the thickness of the skull. fMRI, CT and anatomic images may be co-registered using a stereotactic navigation system (for example Medtronic Stealth Navigation system commercially available from Medtronic, U.S.A. may be used for co-registration). The optimal implantation site will be determined using a combination of these imaging modalities. As an example, anatomic MRI imaging may be used to identify the dorsolateral prefrontal cortical region. Additionally, resting state or task based fMRI may be used to further refine the location for ICI implantation within the anatomical region.

An exemplary operative placement of the ICI(s) may be performed as follows: the patient may be brought into the operating room and induced under general anesthesia. Once the implantation site is identified in imaging space, the implantation location may be localized on the patient's head using a stereotactic navigation system. The implantation site may then be prepared and draped in standard surgical fashion. The skin of the scalp may be infiltrated with a local anesthetic and a 1 cm incision may be made. A small retractor may be placed in the incision and the surface of the skull is exposed. An opening in the outer table of the calvarial bone may then be made (by drilling and/or burring or any other suitable surgical method to accommodate the dimensions and specific structure of the ICI being used (as described hereinabove for the various different types of implants). The bone thickness at the site planned for implantation may be determined from the CT scan of the skull performed prior to implantation.

For example, for the implant 70 illustrated in FIG. 10, the hollow passage 7D made in the calvarial bone may reach the outer surface 6E of the inner table 6. Alternatively, the hollow passage 7D may be formed to a depth of about 2 mm superficial to the outer surface 6C of the inner table 6. It is however noted that the above dimensions are given by way of example only and the depth of the hollow passage 7D may be larger or smaller than the above exemplary depth.

In another example, when implanting the ICI 82 of FIG. 24, the hollow passage 7E may extend to the outer surface 6B of the inner table 6. The ICI 82 is inserted into the hollow passage 7E such that the entire ICI 82 is disposed within the cancellous bone 7 (the diploe) and located between the plane of the outer surface 6B of the inner table 6 and the plane of the inner surface 5B of the outer table 5. After placement of the ICI 82 the shim 190 is inserted into the hollow passage 7E such that the outer surface 190A of the shim 190 is flush with the outer surface 5A of the outer table 5. The shim 190 is then secured and sealed in place with a biocompatible sealant 55 (such as, for example, dental acrylic or any other suitable biocompatible glue or sealant after setting of the sealant 55, the induction coil 186 is suitably connected to the pair of electrically conducting insulated wires 189.

If necessary, depending on the type of implant being used, one or more elongated channels may be formed in the cancellous bone 7 (for example, by drilling or laser ablation as disclosed in detail hereinabove) that are contiguous with the hollow passage 7A (or with the main recess or chamber formed in the calvarium) the channel(s) may then receive of flexible linear electrode arrays that are inserted therein such as, for example, the elongated electrode carrying member 52 of FIG. 8 or the flexible or bendable electrode carrying member 62 of FIG. 9.

If the ICI is similar to the ICI 82 of FIG. 24, after the ICI is properly inserted into the hollow passage 7E, the implant will be slightly recessed relative to the surface of the skull. To accommodate the differences in skull thickness the shim 190 which attaches to the top of the ICI 82 may be selected to be of such a thickness to ensure that the upper surface 190A of the selected shim 190 is flush with the outer surface 5A of the outer table 5. After attaching the ship 190 to the ICI 82, the shim 190 may be securely attached to the outer table by a biocompatible glue or sealant 55 as disclosed in detail hereinabove. Once the ICI is implanted within and attached to the skull. The wound may be irrigated and the scalp 109 may surgically be closed.

It is noted that in the ICIs of the present application, the reference electrode may be disposed in the vicinity of or in contact with the inner table 6 such as, for example, the electrode 13B of the ICI 10 of FIG. 3, and may operate as a reference electrode during sensing/recording.

It is appreciated that certain features of the in invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An intra-calvarial implant (ICI) comprising:
One or more electrodes for sensing electrical signals from a brain of a mammal and for electrically stimulating one or more regions of the brain, the one or more electrodes are configured to be implanted between an outer table and an inner table of the calvarial bone without fully penetrating the inner table;
at least one reference electrode;
an electronic circuitry module operatively connected to the one or more electrodes and to the at least one reference electrode and configured for controlling the sensing and the stimulating, for at least partially processing sensed electrical signals to obtain data for storing the data and for wirelessly transmitting the data to an external receiver; and
a power harvesting device suitably electrically connected to one or more components of the electronic circuitry module of the ICI for energizing one or more components of the ICI.

2. The ICI according to claim 1, wherein the one or more electrodes are attached to an electrode supporting member.

3. The ICI according to claim 1, wherein the electrode supporting member is selected from,
a housing wherein the one or more electrodes are attached to the housing or a part thereof, the housing sealingly includes the electronic circuitry module therein,
an electrode supporting member separate from a housing of the ICI wherein the one or more electrodes are attached to the electrode supporting member and electrically connected to the electronic circuitry module that is sealingly disposed within the housing,
an elongated lead-like flexible electrode supporting member attached to a housing sealingly enclosing the electronic circuitry module, wherein the one or more electrodes are attached to the elongated flexible electrode member, and
a flexible electrode array supporting member electrically connected to a housing sealingly enclosing the electronic circuitry module.

4. The ICI according to claim 1, wherein the electronic circuitry module is selected from,
an electronic circuitry module sealing enclosed in a housing, the housing is disposed in its entirety between the outer table and the inner table of the calvarial bone,
an electronic circuitry module sealingly enclosed within a housing, wherein a part of the housing is disposed between the outer table and the inner table and another part of the housing is disposed outside the outer table, and
an electronic circuitry module sealingly enclosed within a housing disposed outside an outer surface of the outer table.

5. The ICI according to claim 1, wherein the one or more electrodes are disposed in one or more hollow passages formed within a cancellous bone layer of the calvarial bone.

6. The ICI according to claim 1, wherein one or more of the one or more electrodes and the electronic circuitry module are disposed within a chamber formed within a cancellous bone of the calvarial bone.

7. The ICI according to claim 6, wherein the chamber comprises a recess formed within the inner table of the calvarial bone without fully penetrating the inner table.

8. The ICI according to claim 7, wherein the chamber has a chamber opening formed within the outer table, the chamber opening has a cross sectional area and wherein the chamber is selected from,
a chamber in which at least one cross-sectional area of the chamber taken parallel to the cross sectional area of the opening is larger than the cross-sectional area of the chamber opening,
a chamber in which at least one cross-sectional area of the chamber taken parallel to the cross sectional area of the opening is equal to the cross-sectional area of the opening,
a chamber in which at least one cross-sectional area of the chamber taken parallel to the cross sectional area of the opening is smaller than the cross-sectional area of the opening, and
a chamber having at least one laterally extending elongated hollow passage formed within the cancellous bone.

9. The ICI according to claim 1, wherein the one or more electrodes are selected from the list consisting of, multiple single electrodes, a single electrode array, multiple electrode arrays, a flexible electrode array, a foldable electrode array, a rigid electrode array, a planar electrode array, a linear electrode array, a flexible linear electrode array, a curved surface electrode array, an electrocorticography (Ecog) electrode array and any non-mutually exclusive combinations thereof.

10. The ICI according to claim 1, wherein the electronic circuitry module is selected from,
an electronic circuitry module programmed for wirelessly receiving control signals from an external control unit, selecting a subset of electrodes from the one or more electrodes responsive to the received control signals, sensing electrical brain signals through the selected electrode(s), conditioning and/or amplifying the signals and wirelessly communicating the signals to an external receiver,
an electronic circuitry module programmed for wirelessly receiving control signals from an external control unit, selecting a subset of electrodes from the one or more electrodes responsive to the received control signals and stimulating through the selected subset of electrodes one or more regions of the brain,
an electronic circuitry module programmed for wirelessly receiving control signals from an external control unit, selecting a first subset of electrodes from the one or more electrodes responsive to the received control signals, sensing electrical brain signals through the first subset of electrodes, conditioning and/or amplifying the signals and wirelessly communicating the signals to an external receiver, wirelessly receiving control signals from an external control unit, selecting a second subset of electrodes from the one or more electrodes responsive the control signals and stimulating through the second subset of electrodes one or more regions of the brain, and
an electronic circuitry module programmed for selecting a first subset of electrodes from the one or more electrodes, sensing electrical brain signals through the selected first subset of electrodes, conditioning and/or amplifying the signals, processing the signals to detect an indication associated with a neurological and/or neuropsychiatric and/or psychiatric state of the brain, selecting a second subset of electrodes from the one or more electrodes based on the indication and stimulating through the second subset of electrodes one or more regions of the brain.

11. The ICI according to claim 10, wherein the first subset and the second subset are selected from,
the first subset is different than the second subset, and
the first subset is identical to the second subset.

12. The ICI according to claim 1, wherein the power harvesting device is selected from an ultrasonic energy harvesting device and an electromagnetic power harvesting device.

13. The ICI according to claim 1, wherein the power harvesting device is selected from a power harvesting device comprising an induction coil and a power harvesting device comprising a piezoelectric transducer.

14. The ICI according to claim 1, wherein at least one part of the power harvesting device is disposed under or within the scalp of the mammal.

15. The ICI according to claim 1, wherein at least one part of the power harvesting device is disposed within the ICI.

16. The ICI according to claim 2, wherein the electrode supporting member has a shape selected from, a disc-like shape, a cylindrical shape, a shape having an ellipsoidal cross section, a shape having a polygonal cross section, and a shape having an irregular cross section.

17. The ICI according to claim 2, wherein the electrode supporting member is configured as an openable and closable housing having an openable and closable compartment therein.

18. (canceled)

19. The ICI according to claim 1, wherein the mammal is selected from, a non-human mammal, and a human patient.

20. The ICI according to claim 1, wherein the at least one reference electrode is selected from, at least one reference electrode disposed between the outer table and the inner table of the calvarial bone without fully penetrating the inner table, and
at least one reference electrode disposed on the electrode supporting member,
at least one reference electrode disposed within the outer table or on the outer surface of the outer table,
at least one reference electrode disposed between the outer surface of the outer table and the scalp, and
at least one reference electrode disposed under or within the scalp.

21. The ICI according to claim 3, wherein the at least one reference electrode is selected from,
at least one reference electrode attached to the electrode supporting member,
at least one reference electrode attached to the housing or to any parts thereof,
at least one reference electrode attached to an electrode supporting member separate from a housing of the ICI,
at least one reference electrode attached to an elongated lead-like flexible electrode supporting member attached to a housing sealingly enclosing the electronic circuitry module, wherein the one or more electrodes are attached to the elongated flexible electrode member, and
at least one reference electrode attached to a flexible electrode array electrically connected to a housing sealingly enclosing the electronic circuitry module.

22. An ICI system, comprising:
One or more of the intra-calvarial implant (ICI) of claim 1 for sensing electrical activity in a brain and/or for stimulating the brain,
an external controller unit configured for wirelessly bidirectionally communicating with the one or more ICI to receive and/or transmit signals to the one or more ICI.

23-32. (canceled)

33. A method for implanting an intra-calvarial implant (ICI) of claim 1 in a mammal, the method comprising the steps of:
making an incision in a scalp of the mammal,
exposing a region of the calvarial bone of the mammal,
forming one or more openings in an outer table of the calvarial bone,
forming one or more hollow passages in the cancellous bone of the calvarial bone without fully penetrating an inner table of the calvarial bone,
inserting at least part of the ICI into the one or more hollow passages such that the one or more electrodes of the ICI are disposed between the outer table and the inner table of the calvarial bone, and
arranging the scalp to cover the exposed region of the scalp.

34-44. (canceled)
